(19) United States
(12) Patent Application Publication
(10) Pub. No.: US 2025/0135086 A1
(43) Pub. Date: May 1, 2025

(54) VALVE ASSEMBLIES FOR OPHTHALMIC SURGICAL CASSETTES

(71) Applicant: Alcon Inc., Fribourg (CH)

(72) Inventors: Behzad HASHEMI, Irvine, CA (US); Ali NIKKHAH, Mesa, AZ (US); Steven Benjamin ROLL, San Clemente, CA (US); Ming LU, Placentia, CA (US); Vincent A. BAXTER, Temecula, CA (US)

(21) Appl. No.: 18/914,645

(22) Filed: Oct. 14, 2024

Related U.S. Application Data
(60) Provisional application No. 63/594,906, filed on Oct. 31, 2023.

(51) Int. Cl. A61M 1/00; A61F 9/007

(52) U.S. Cl. CPC A61M 1/72; A61F 9/007

(57) ABSTRACT

Certain embodiments disclosed herein provide a surgical cassette including a housing, one or more retaining rings coupled to the housing, and one or more valve assemblies coupled to the housing by the one or more retaining rings. The one or more valve assemblies configured to control fluid communication between one or more channels within the housing. Each valve assembly includes a valve body having a first end with a first shoulder, a second end with a second shoulder, a cylindrical surface connecting the first shoulder and second shoulder, and an upper valve elastomer. The upper valve elastomer includes a first side configured to engage with the first shoulder of the valve body and a second side configured to engage with the retaining ring to facilitate a fluidic seal at a base of the housing.

### CROSS-REFERENCE TO RELATED APPLICATIONS

This application claims the benefit of U.S. Provisional Application Ser. No. 63/594,906 (filed on Oct. 31, 2023), the content of which is incorporated herein by reference in its entirety.

### INTRODUCTION

Cataract surgery involves removing a cataractous lens and replacing the lens with an artificial intraocular lens (IOL). The cataractous lens is typically removed by fragmenting the lens and aspirating the lens fragments out of the eye. The lens may be fragmented using, e.g., a phacoemulsification probe, a laser probe, or another suitable instrument. During the procedure, the probe fragments the lens, and the fragments are aspirated out of the eye through, e.g., a hollow needle or cannula. Throughout the procedure, irrigating fluid is pumped into the eye to maintain an intraocular pressure (IOP) and prevent collapse of the eye.

During cataract surgery, a surgical cassette having one or more peristaltic and/or venturi pumps and one or more valve assemblies may be operably coupled with a fluidics module of a surgical console and used to facilitate the aspiration and irrigation functionalities described above. In general, the one or more valve assemblies of the surgical cassette are operable to control the application of flow generated by the one or more peristaltic pumps during the surgical procedure.

However, conventional surgical cassettes have a number of significant shortcomings including, for example, relatively tight sealing tolerances, relatively high torque required for valve operation, restricted flow capacity, the inability to maintain a secure connection with the surgical console, relatively high fabrication costs and complexity, and redundancy issues, among others.

### BRIEF SUMMARY

The present disclosure relates generally to ophthalmic surgical cassettes, valve assemblies therefor, and methods of use thereof.

In certain embodiments, a surgical cassette is provided for use in ophthalmic surgical procedures. The surgical cassette includes a housing having a partition separating a first surface of the housing from a second surface of the housing, one or more ports formed in the first surface of the housing, and one or more corresponding channels adjoining the second surface of the housing and in fluid communication with the one or more ports. The surgical cassette includes one or more valve assemblies coupled to the housing and configured to control fluid communication between the one or more channels of the housing. Each of the one or more valve assemblies includes a valve body having a first end with a first shoulder, a second end with a second shoulder, a cylindrical surface connecting the first shoulder and the second shoulder, and an upper valve elastomer. The upper valve elastomer includes a first side configured to engage with the first shoulder of the valve body and a second side configured to engage with the retaining ring to facilitate a fluidic seal at a base of the housing. The valve body is rotatable about a first axis orthogonal to the first end and relative to the first surface of the housing to align one or more passages with the one or more ports of the housing to open fluid communication between the one or more corresponding channels. A drive interface is formed on the first end of the valve body and configured to engage a drive mechanism for rotating the valve body.

In certain embodiments, a surgical cassette is provided for use in ophthalmic surgical procedures. The surgical cassette includes a housing having a partition separating a first surface of the housing from a second surface of the housing, one or more ports formed in the first surface of the housing, and one or more corresponding channels adjoining the second surface of the housing and in fluid communication with the one or more ports. The surgical cassette includes one or more valve assemblies coupled to the housing and configured to control fluid communication between the one or more channels of the housing. Each of the one or more valve assemblies includes a valve body having a first end with a first shoulder, a second end with a second shoulder, a cylindrical surface connecting the first shoulder and the second shoulder, and a biasing member. The biasing member is disposed along the cylindrical surface of the valve body and configured to engage with the retaining ring to facilitate a fluidic seal at a base of the housing. The valve body is rotatable about a first axis orthogonal to the first end and relative to the first surface of the housing to align one or more passages with the one or more ports of the housing to open fluid communication between the one or more corresponding channels. A drive interface is formed on the first end of the valve body and configured to engage a drive mechanism for rotating the valve body.

In certain embodiments, a surgical cassette is provided for use in ophthalmic surgical procedures. The surgical cassette includes a housing having a partition separating a first surface of the housing from a second surface of the housing, one or more ports formed in the first surface of the housing, and one or more corresponding channels adjoining the second surface of the housing and in fluid communication with the one or more ports. The surgical cassette includes one or more valve assemblies coupled to the housing and configured to control fluid communication between the one or more channels of the housing. Each of the one or more valve assemblies includes a valve body having a first end with a first shoulder, a second end with a second shoulder, a cylindrical surface connecting the first shoulder and the second shoulder, a first sealing mechanism, and a second sealing mechanism. The first sealing mechanism is configured to engage with the retaining ring to facilitate a seal at a base of the housing. The second scaling mechanism is configured to provide the seal at the base of the housing. The valve body is rotatable about a first axis orthogonal to the first end and relative to the first surface of the housing to align one or more passages with the one or more ports of the housing to open fluid communication between the one or more corresponding channels. A drive interface is formed on the first end of the valve body and configured to engage a drive mechanism for rotating the valve body.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIG. 2A is a back side isometric view of an example surgical cassette which may be operably coupled to a console of an ophthalmic surgical system, according to certain embodiments.

FIG. 2B is a back side elevation view of the surgical cassette of FIG. 2A, according to certain embodiments.

FIG. 2C is an exploded front side isometric view of the surgical cassette of FIG. 2A, according to certain embodiments.

FIG. 2D is an exploded back side isometric view of the surgical cassette of FIG. 2A, according to certain embodiments.

FIG. 2E is a front side elevation view of a base of the surgical cassette of FIG. 2A, according to certain embodiments.

FIGS. 3A-3B are enlarged exploded front side and back side isometric views, respectively, of a portion of the surgical cassette of FIG. 2A illustrating an example valve assembly having an upper valve elastomer, according to certain embodiments.

FIG. 4A is a top side isometric view illustrating an example valve assembly having an upper valve elastomer, according to certain embodiments.

FIG. 4B is a bottom side isometric view illustrating the valve assembly seen in FIG. 4A, according to certain embodiments.

FIG. 4C is a bottom up perspective view illustrating the valve assembly seen in FIG. 4A, according to certain embodiments.

FIG. 4D is a top down perspective view illustrating the valve assembly seen in FIG. 4A, according to certain embodiments.

FIG. 4E is a cross sectional side view illustrating the valve assembly seen in FIG. 4A, according to certain embodiments.

FIG. 5A is a cross sectional side view illustrating the valve assembly seen in FIGS. 4A-4E, disposed in the surgical cassette of FIG. 2A, according to certain embodiments.

FIG. 5B is a magnified view of FIG. 5A illustrating a cross sectional view of the upper valve elastomer of the valve assembly, according to certain embodiments.

FIGS. 6A-6B are enlarged exploded front side and back side isometric views, respectively, of a portion of the surgical cassette of FIG. 2A illustrating another example valve assembly having a biasing member, according to certain embodiments.

FIG. 7A is a top side isometric view illustrating an example valve assembly having a biasing member, according to certain embodiments.

FIG. 7B is a bottom side isometric view illustrating the valve assembly seen in FIG. 7A, according to certain embodiments.

FIG. 7C is a bottom up perspective view illustrating the valve assembly seen in FIG. 7A, according to certain embodiments.

FIG. 7D is a top down perspective view illustrating the valve assembly seen in FIG. 7A, according to certain embodiments.

FIG. 7E is a cross sectional side view illustrating the valve assembly seen in FIG. 7A, according to certain embodiments.

FIG. 8A is a cross sectional side view illustrating the valve assembly seen in FIGS. 7A-7E, disposed in the surgical cassette of FIG. 2A, according to certain embodiments.

FIG. 8B is a magnified view of FIG. 8A illustrating a cross sectional view of the biasing member of the valve assembly, according to certain embodiments.

FIGS. 9A-9B are front side elevation views of a portion of the surgical cassette of FIG. 2A illustrating two different valve positions, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes, valve assemblies therefor, and methods of use thereof.

Certain embodiments disclosed herein provide valve assemblies for a surgical cassette with improved sealing function. Current valves for surgical cassettes typically have elastomeric seals disposed at one end (e.g., a lower end) of the valves, which interact with rigid materials on the surgical cassettes or other components integrated therewith. When the valves are compressed, the elastomeric seals act to seal one or more ports, or fluid channels, of the surgical cassettes, as partially caused by reactive forces of the elastomeric seals in response to valve compression. However, the sealing forces provided by the elastomeric seals may vary from surgical cassette to surgical cassette, based on differing tolerances between each valve, elastomeric seal, and corresponding surgical cassette. Additionally, such sealing forces may decrease over time due to compression set or stress relaxation (e.g., fatigue or loss of springiness), which results from prolonged periods of valve compression. This variability and/or reduced efficacy in sealing by the valves may lead to reduced overall performance and reliability of the surgical cassette.

To overcome the drawbacks described above, embodiments herein disclose valve assemblies having both lower and upper valve elastomers configured to fluidically seal the valve assemblies at a base of a surgical cassette housing. Because sealing forces are provided by elastomers disposed at both lower and upper ends of the valve assemblies, compression set and stress relaxation become less of a concern, and such sealing forces may be maintained for longer periods of time, thereby facilitating increased lifespan and/or service life of surgical cassettes.

Further, the dependence of such sealing forces on the tolerances between the valve assemblies, elastomeric seals, and corresponding surgical cassettes may be minimized. Still further, at least in part due to the second, upper valve elastomer being separate from a sealing interface on the opposing, lower end of the valve assembly, embodiments disclosed herein provide valve assemblies having greater design and material flexibility for the elastomeric seals. The improved sealing capabilities of the valve assemblies described herein directly impact the overall performance of the surgical cassette, leading to more accurate and consistent fluid movement, which is essential for patient safety and surgical success during ophthalmic surgical procedures.

Certain embodiments described herein provide a valve assembly with a valve body having a first end with a first shoulder, a second end with a second shoulder, and a cylindrical surface connecting the first shoulder and the second shoulder, a first sealing mechanism, and a second sealing mechanism. The first sealing mechanism is configured to engage with the retaining ring to facilitate a seal at a base of the housing. As described herein, the “first sealing mechanism” comprises an upper valve elastomer, a biasing member, or other mechanism suitable for facilitating the seal at the base of the housing. As an example, the “biasing member” may be a spring that can provide a biasing force (e.g., downwards force) to create the seal at the base of the housing. The second sealing mechanism is configured to provide a seal at the base of the housing and minimize ballooning into ports at a base of a housing. As described herein, the “second sealing mechanism” comprises a lower valve elastomer or other mechanism suitable for providing the seal at the base of the housing.

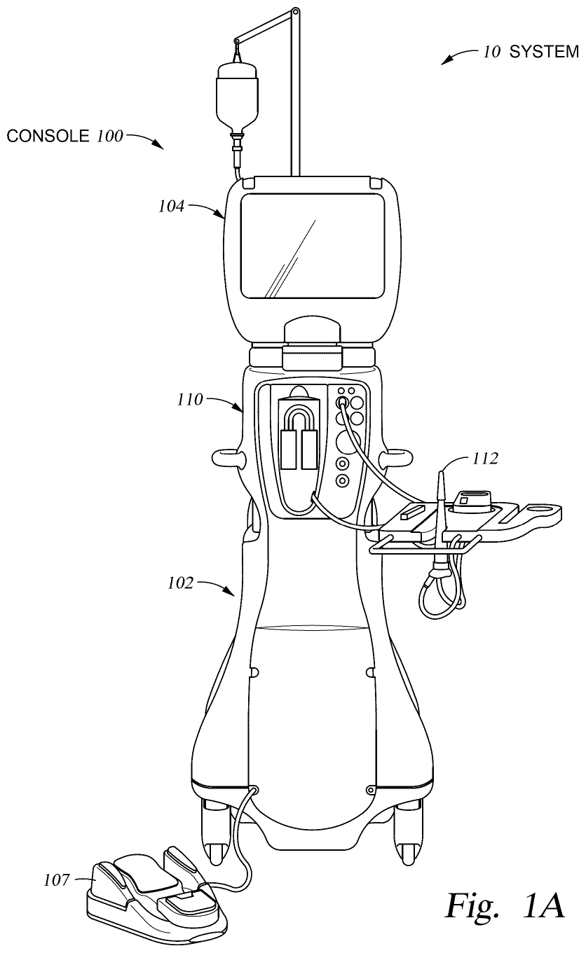

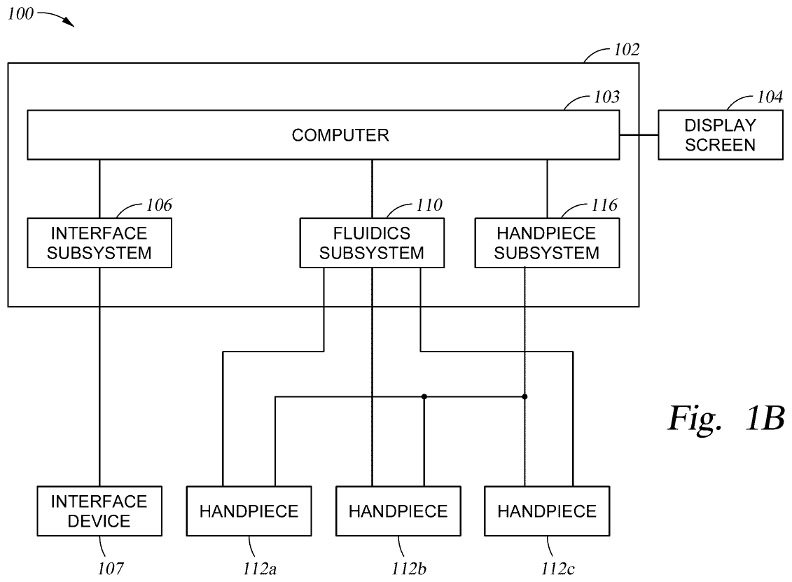

FIG. 1A illustrates an example of an ophthalmic surgical system 10 that may be used to perform ophthalmic procedures on an eye, according to certain embodiments. In the illustrated embodiments, system 10 includes console 100 (also referred to as a “surgical console”), an interface device 107 (e.g., a foot pedal), and a handpiece 112. Console 100 includes a housing 102, a display screen 104, and a fluidics subsystem 110. The components of system 10 and console 100 may be coupled as shown and described in more detail with reference to FIG. 1B.

FIG. 1B illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, and 116, which support interface device 107 and handpieces 112 (112a-c). An interface device 107 receives input to console 100, sends output from console 100, and/or processes the input and/or output. Examples of an interface device 107 include a foot pedal, manual input device (e.g., a keyboard), and a display. Interface subsystem 106 receives input from and/or sends output to interface device 107.

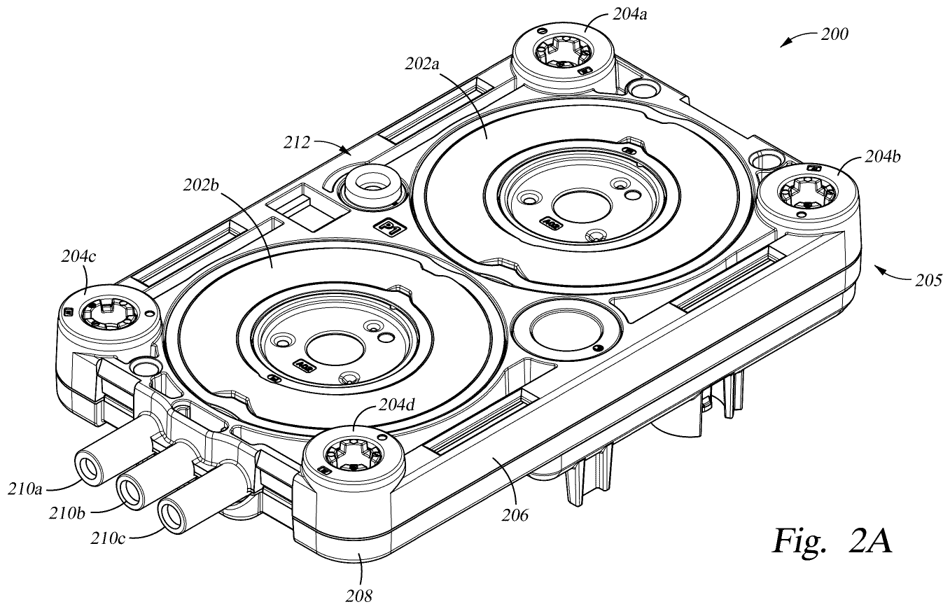

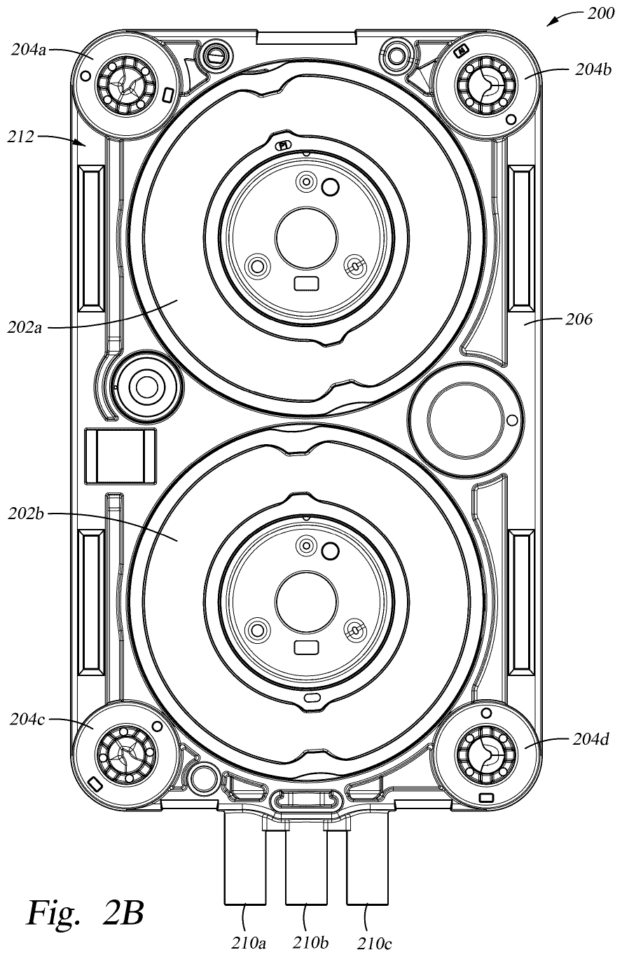

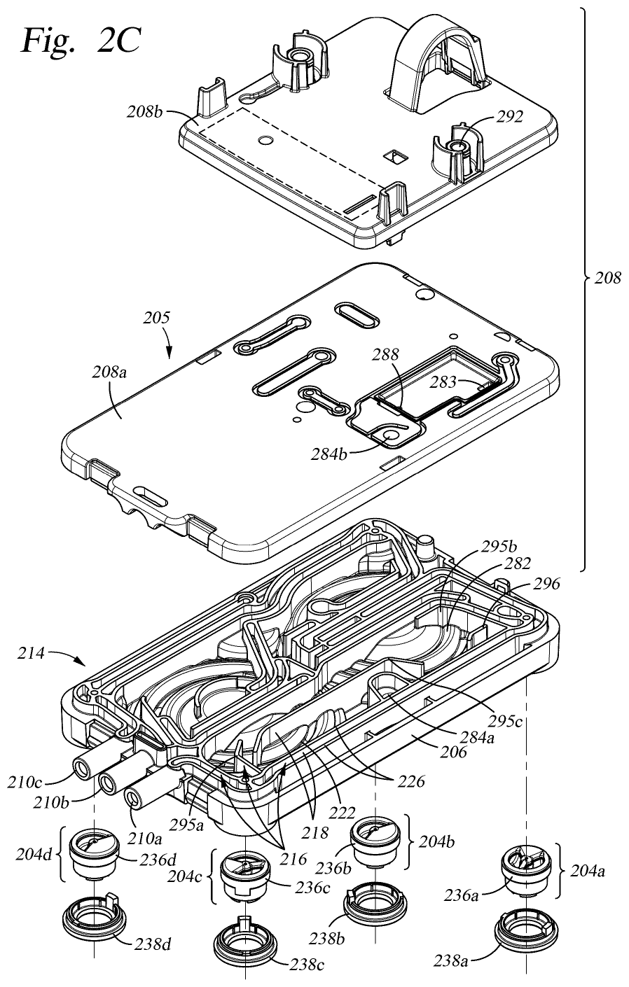

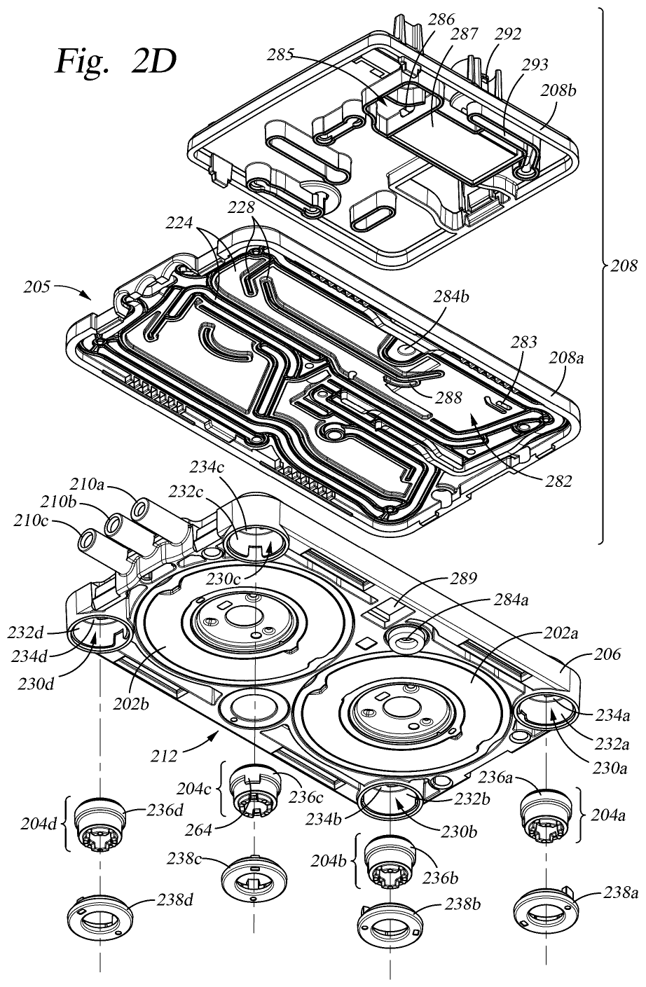

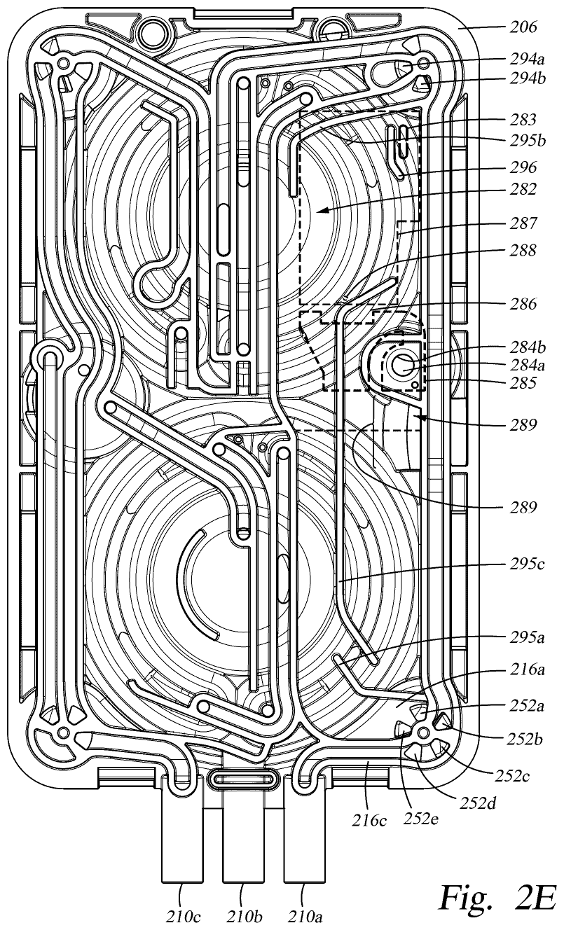

Fluidics subsystem 110 provides fluid control for one or more handpieces 112 (112a-c). For example, fluidics subsystem 110 may manage fluid for an irrigating cannula. In some embodiments, fluidics subsystem 110 may be operatively coupled to a surgical cassette (e.g., surgical cassette 200 of FIGS. 2A-2B). For example, surgical cassette 200 may be inserted into, attached to, and/or integrated with fluidics subsystem 110 via a coupling mechanism. The coupling mechanism may comprise one or more of a latching mechanism, locking mechanism, or other similar connection mechanism. When fluidics subsystem 110 is operatively coupled to surgical cassette 200, fluidics subsystem 110 may control irrigation and/or aspiration of fluids through surgical cassette 200.

Handpiece 112 may be any suitable ophthalmic surgical instrument, e.g., an ultrasonically-driven phacoemulsification (phaco) handpiece, a laser handpiece, an irrigating cannula, a vitrectomy handpiece, or another suitable surgical handpiece. Handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece. Computer 103 controls operation of ophthalmic surgical system 10. In certain embodiments, computer 103 includes a controller that sends instructions to components of system 10 to control system 10. A display screen 104 shows data provided by computer 103.

FIG. 2A is a back side isometric view of an example surgical cassette 200 which may be operably coupled to a console of an ophthalmic surgical system (e.g., console 100 of ophthalmic surgical system 10 illustrated in FIGS. 1A-1B), according to certain embodiments. For example, as described above, surgical cassette 200 may be operatively coupled to fluidics subsystem 110 of console 100. FIG. 2B is a back side elevation view of surgical cassette 200 of FIG. 2A, according to certain embodiments. FIGS. 2A-2B are described together herein for clarity.

In some embodiments, the general operations of the surgical cassette 200 are described in more detail in U.S. Patent Application No. 63/175,589 filed on Apr. 16, 2021, entitled “Systems and Methods for Post-Occlusion Break Surge Mitigation”, which is hereby incorporated by reference in its entirety (other manners of surgical cassette operation are also contemplated). Surgical cassette 200 includes two pump assemblies 202 (202a-b) which provide a source of pressure and/or vacuum and four valve assemblies 204 (204a-d) which control pressure and/or fluid communication within surgical cassette 200. In certain other embodiments, there may be only one pump assembly or more than two pump assemblies. In certain other embodiments, there may be more or less than four valve assemblies (e.g., 2-6 valve assemblies).

In certain embodiments, an external source of pressure and/or vacuum is coupled to surgical cassette 200. In such embodiments, the external source may be either in place of or in addition to pump assemblies 202.

Surgical cassette 200 has a housing 205 including a base 206, a cover assembly 208 coupled to base 206, and inlet/outlet ports 210 (210a-c) in base 206 which provide pressure and/or fluid communication between inside and outside of the housing 205. In some embodiments, flow lines (e.g., tubing) may also be coupled between each port 210a-c and a corresponding component of fluidics subsystem 110 and/or a corresponding handpiece 112a-c (shown in FIGS. 1A-1B).

In certain embodiments, one of a first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump 202b assembly are identical to each other.

Valve assemblies 204 are coupled to base 206. Valve assemblies 204 function cooperatively to control pressure and/or fluid communication within and through surgical cassette 200. In the illustrated embodiments, surgical cassette 200 includes a first valve assembly 204a, a second valve assembly 204b, a third valve assembly 204c, and a fourth valve assembly 204d. As shown, in the embodiments of FIG. 2A, the four valve assemblies 204 are arranged at the four corners of housing 205 and surrounding the two pump assemblies 202 which are arranged towards a center of housing 205. However, in certain other embodiments, pump assemblies 202 and valve assemblies 204 may have any other suitable arrangement.

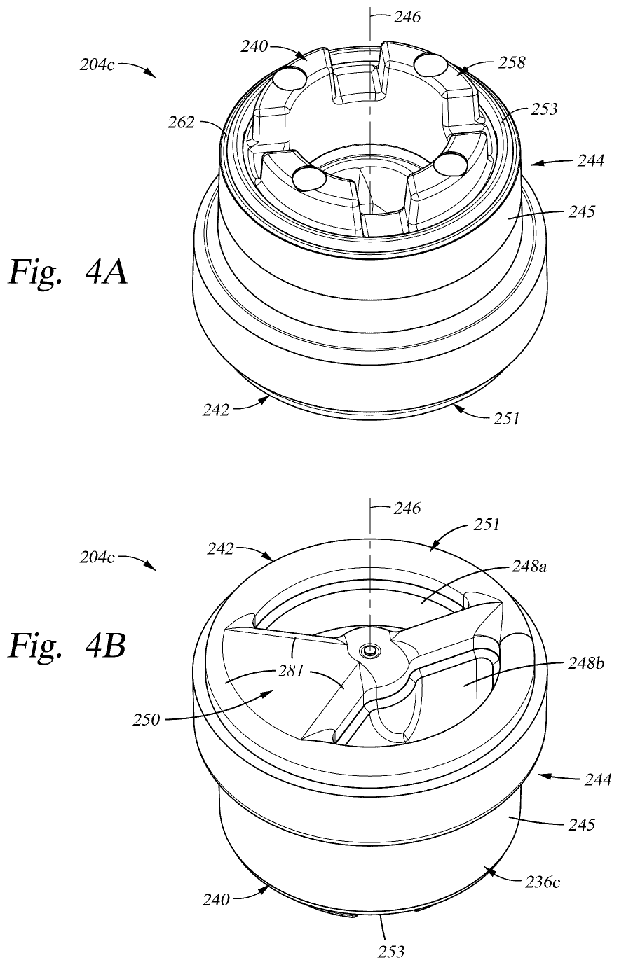

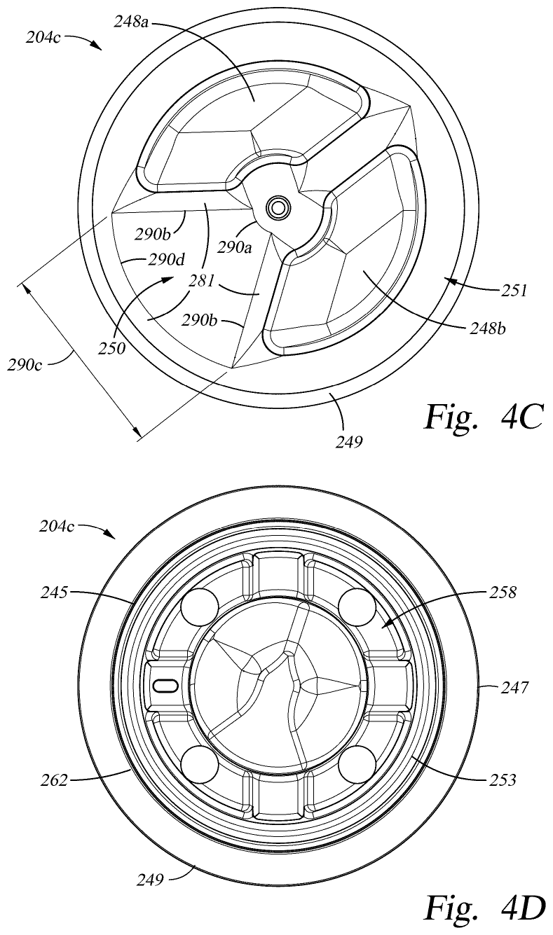

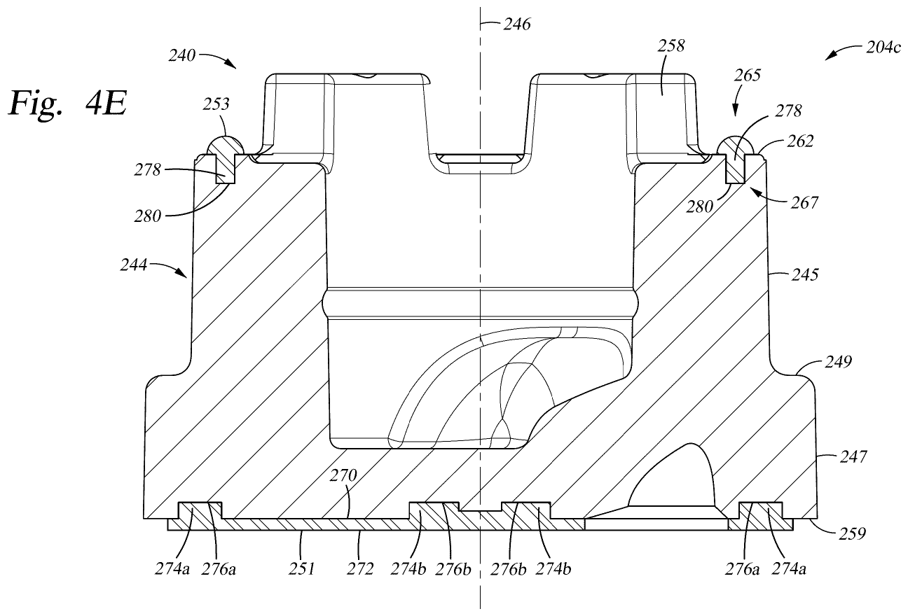

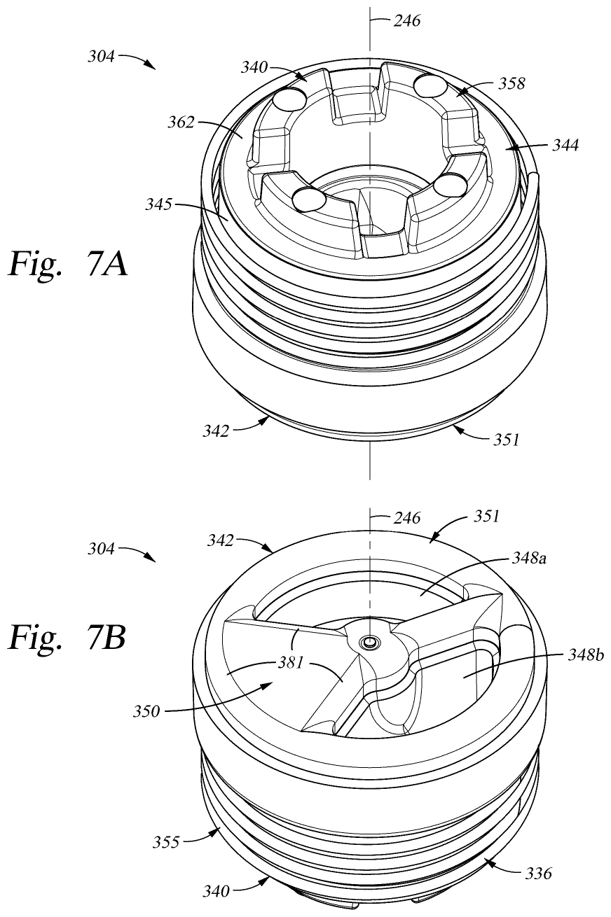

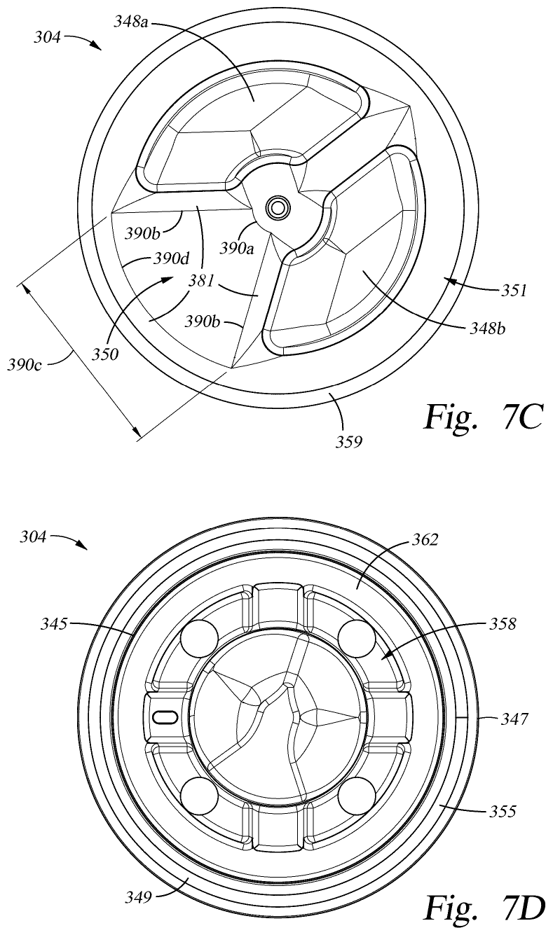

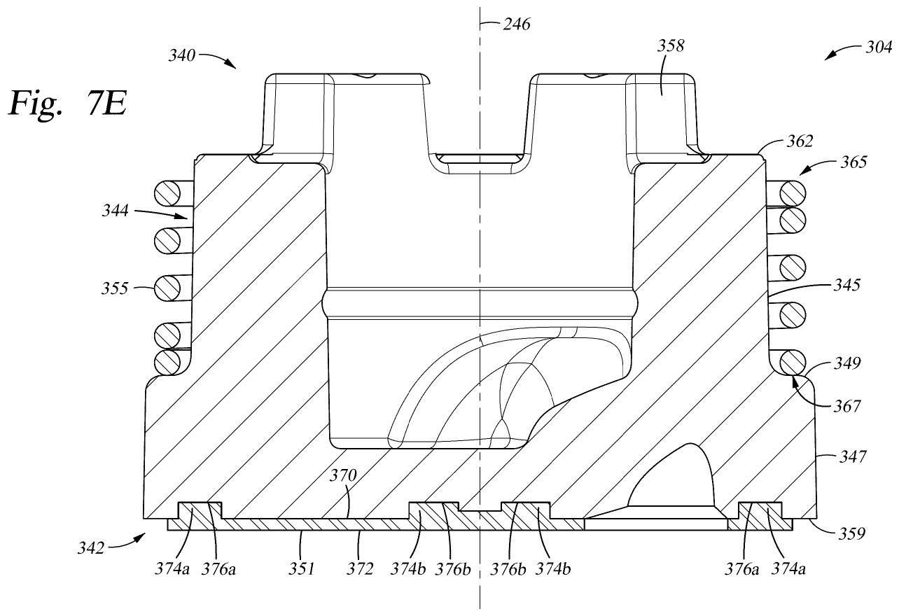

In certain embodiments, valve assemblies 204 may be operated to route fluid flow selectively between one or more channels of housing 205 as described in more detail below with respect to FIGS. 4A-4E. Alternative valve assemblies which may be implemented in surgical cassette 200 are described in more detail below with reference to FIGS. 7A-7E. In some embodiments, first valve assembly 204a and third valve assembly 204c may be in pressure and/or fluid communication with first pump assembly 202a and port 210a to provide aspiration (suction) through port 210a during an operation, and second valve assembly 204b and fourth valve assembly 204d may be in pressure and/or fluid communication with second pump assembly 202b and port 210c to provide irrigation (infusion) through port 210c during the same operation. In certain embodiments, port 210b may be an auxiliary port used for other operational purposes (e.g., servicing surgical cassette 200).

Pump assemblies 202 and valve assemblies 204 are located on a back side 212 of base 206 which is visible in FIGS. 2A-2B. Cover assembly 208 is coupled to a front side 214 (shown in FIG. 2C) of base 206 which faces away from back side 212. In certain embodiments, cover assembly 208 may be welded, bonded, or fastened to base 206 using any suitable coupling mechanism. For example, cover assembly 208 may be coupled to base 206 using a solid-state welding technique (e.g., ultrasonic welding in which high-frequency ultrasonic acoustic vibrations are locally applied to work parts being held together under pressure to create a solid-state weld).

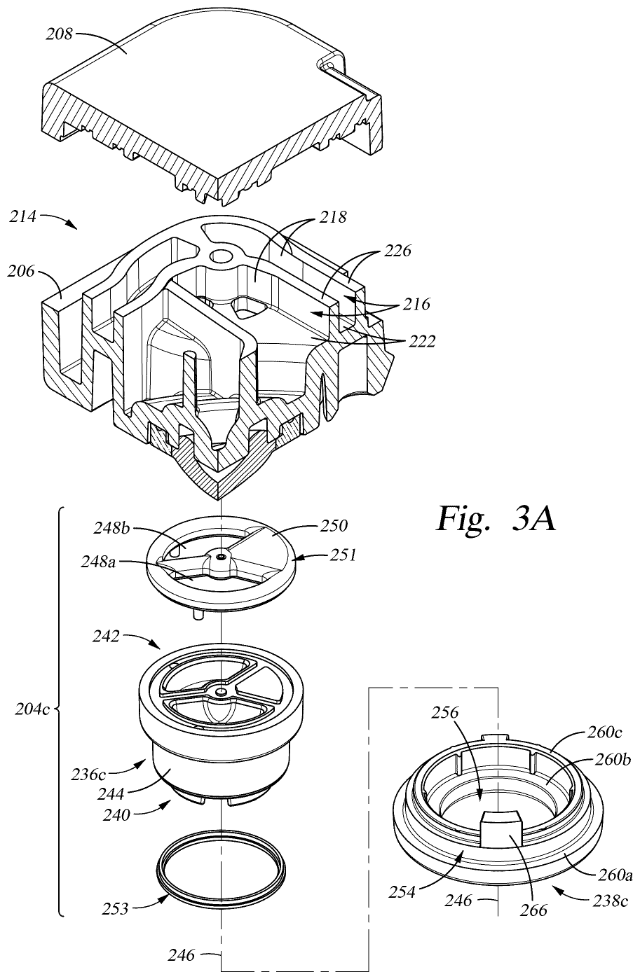

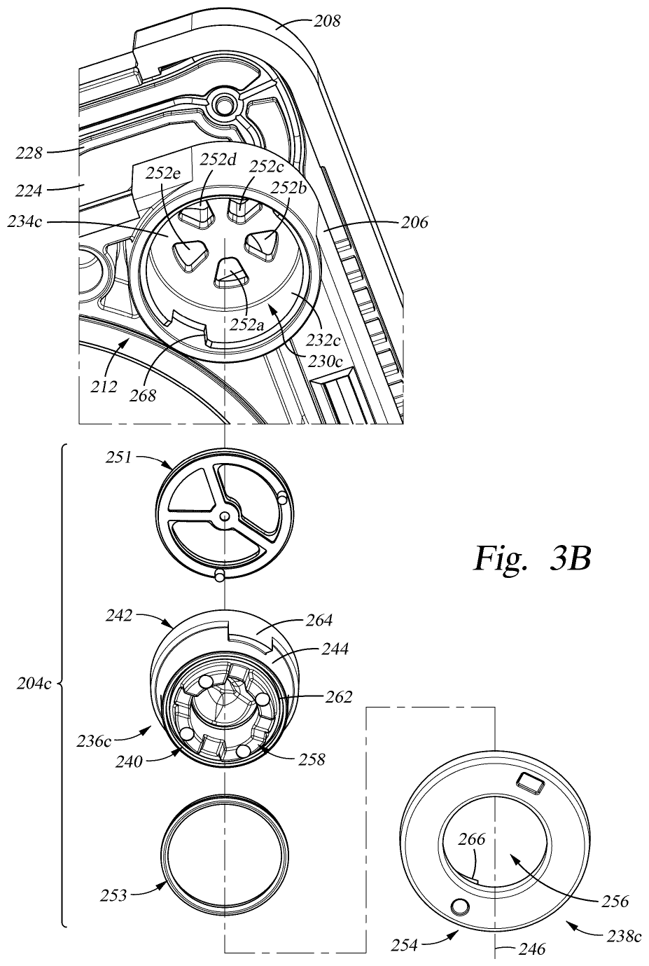

Back side 212 of base 206 is configured to interface with console 100 when surgical cassette 200 is coupled thereto. For example, a drive interface on a valve body of each valve assembly 204 may engage a corresponding drive mechanism of console 100 for rotating the corresponding valve body. The valve body is described in more detail below with respect to FIGS. 3A-3B. In certain embodiments, the drive mechanism is a direct drive motor which operates at lower torque with faster valve response time when compared to a geared drive motor which is conventionally used. However, the embodiments described herein may use any suitable type of drive motor. The components of surgical cassette 200 are described in more detail below with respect to FIGS. 2C-2D.

FIG. 2C is an exploded front side isometric view of surgical cassette 200 of FIG. 2A, according to certain embodiments. FIG. 2D is an exploded back side isometric view of surgical cassette 200 of FIG. 2A, according to certain embodiments. FIGS. 2C-2D are described together herein for clarity. In the illustrated embodiments, cover assembly 208 includes two separate pieces including first cover piece 208a (also referred to as a “cover”) coupled directly to front side 214 of base 206 and second cover piece 208b (also referred to as a “handle”) coupled to cover 208a. In some other embodiments, cover assembly 208 may consist of only a single piece.

A plurality of channels 216 are formed in housing 205. The plurality of channels 216 are arranged to provide a plurality of independent fluid paths between pump assemblies 202, valve assemblies 204, and inlet/outlet ports 210. Each channel 216 is longitudinally defined in a first direction parallel to a plane of housing 205. Each channel 216 includes sidewalls 218 oriented perpendicular to the first direction which enclose the corresponding channel 216 therebetween. In addition, a depth of each channel 216 is defined in a second direction perpendicular to the first direction between a lower wall (also referred to as a “partition”) of base 206 (e.g., front side surface 222 thereof) and an inner surface 224 (shown in FIG. 2D) of cover 208a. The plurality of channels 216 are sealingly enclosed through contact between a first surface 226 formed on front side 214 of base 206 and a corresponding face 228 (shown in FIG. 2D) extending from inner surface 224 of cover 208a.

In order to effect sealing, first surface 226 and corresponding face 228 are molded to include matching contours and assembled to ensure precise alignment between opposing sides. In some other embodiments, the plurality of channels 216 may be formed in cover 208a instead of base 206 and sealingly enclosed through contact between a back side surface of cover 208a and a corresponding front side face of base 206. In some other embodiments, base 206 and cover 208a may be integrally formed as a single piece. In such embodiments, base 206 and cover 208a may be injection molded with the use of a slide technique.

Four bores 230 (230a-d) are formed in back side 212 of base 206 for receiving a corresponding valve assembly 204. Each bore 230 is defined by a cylindrical inner wall 232 (232a-d) and a first side 234 (234a-d). In certain other embodiments, there may be more or less than four bores to correspond to each valve assembly. One or more ports (e.g., five shown in FIGS. 3A-3B) are formed through base 206. The ports connect front side surface 222 and opposite the first side 234 of base 206. Each port corresponds to one of the plurality of channels 216 in contact with, or adjoining, front side surface 222 of base 206. The ports are described in more detail below with respect to FIGS. 3A-3B.

Each valve assembly 204 generally includes a valve body 236 (236a-d), an upper valve elastomer 253 (or first sealing mechanism), and a lower valve elastomer 251 (or second sealing mechanism). Upper valve elastomer 253 is configured to facilitate a fluidic seal provided by lower valve elastomer 251, which is configured to be disposed in a corresponding bore 230. Valve body 236 is coupled to the housing by a retaining ring 238 (238a-d), such that valve body 236 and retaining ring 238 fit together in a stacked arrangement. Valve body 236 is disposed between a first side 234 of base 206 and corresponding retaining ring 238.

Retaining ring 238 applies a sealing force on corresponding valve body 236 to press valve body 236 against first side 234 of base 206 as described in more detail below. In some other embodiments, instead of being defined within base 206, each bore 230 may be defined within a corresponding retaining ring 238 that fits around a corresponding valve body 236. In some other embodiments, each valve body 236 may be rotatably coupled to base 206 using a retention cap. The retention cap may be disposed through valve body 236 (e.g., aligned with a longitudinal axis of valve body 236). In such embodiments, each valve body 236 may be coupled to base 206 without being disposed in a corresponding bore 230.

A venturi reservoir 282 is disposed inside housing 205 of surgical cassette 200 between base 206 and cover 208a. Venturi reservoir 282 serves at least two primary functions that are integral to the operation of surgical cassette 200. In general, venturi reservoir 282 provides a vacuum source for suction of fluids during a venturi operation and provides a fluid volume sink for venting vacuum pressure that may build-up within surgical cassette 200 in the event of a post-occlusion break surge.

FIG. 2E is a front side elevation view of base 206, according to certain embodiments. Note that in FIG. 2E, some parts of cover 208a and handle 208b are shown in phantom for illustrative purposes. Although some aspects of venturi reservoir 282 are visible in FIGS. 2C-2D, certain aspects are illustrated more clearly in FIG. 2E. FIGS. 2C-2E are, therefore, described together herein for clarity.

Surgical cassette 200 may be coupled to an external vacuum source (e.g., a venturi source) disposed in console 100. Vacuum pressure from the external vacuum source is applied to venturi reservoir 282 through a vacuum port 283 in cover 208a. In the illustrated embodiments, vacuum port 283 is an elongated slot. In certain embodiments, vacuum pressure within venturi reservoir 282 is about 720 mmHg (millimeters of mercury). In certain embodiments, maximum air flow through vacuum port 283 is about 1.2 standard liters per minute. The external vacuum source is configured to apply vacuum pressure to venturi reservoir 282 through a vacuum flow path that passes from upstream to downstream through vacuum port 283 in cover 208a and into handle 208b where the air is filtered before passing back through respective openings in cover 208a and base 206 before reaching the external vacuum source. The vacuum flow path is described in more detail below.

In certain embodiments, an opening 284a in back side 212 of base 206 is coupled to a vacuum port on console 100 leading to the external vacuum source. Opening 284b in cover 208a is aligned with opening 284a in base 206. Openings 284a-b are oriented in a direction perpendicular to a plane of base 206. Openings 284a-b are in pressure communication between the external vacuum source and a catch reservoir 285 that is defined between cover 208a and handle 208b. Catch reservoir 285 is in fluid communication, through opening 286, with a filter 287 that is disposed between cover 208a and handle 208b. Catch reservoir 285 catches liquid that leaks through filter 287 and prevents the leaked liquid from entering the vacuum source.

Filter 287 is in pressure communication between the external vacuum source and vacuum port 283 in cover 208a. The location of filter 287 overlays at least a portion of venturi reservoir 282 enabling the use of a more compact housing 205 compared to other designs. An upstream side of filter 287 faces towards cover 208a. A downstream side of filter 287 faces towards handle 208b. Filter 287 seals with handle 208b to prevent liquid from leaking around filter 287. Filter 287 permits air to pass from vacuum port 283 to catch reservoir 285 in handle 208b while blocking liquid from passing to the downstream side of filter 287 and into the external vacuum source. In certain embodiments, filter 287 is directly coupled to handle 208b (e.g., ultrasonically welded). In certain embodiments, filter 287 is hydrophobic, and thus impermeable to aqueous fluids.

In certain cases, liquid is able to pass through vacuum port 283 and becomes trapped between cover 208a and the upstream side of filter 287. A filter drain hole 288 is disposed through cover 208a in a position overlaying a lower end of filter 287. Filter drain hole 288 is in fluid communication between the upstream side of filter 287 and venturi reservoir 282 to drain the trapped liquid back into venturi reservoir 282. Draining of the trapped liquid through filter drain hole 288 helps maintain maximum usable filter area for maximum air flow through vacuum port 283. In certain embodiments, filter drain hole 288 is much smaller than vacuum port 283 in terms of cross-sectional area to prevent or reduce fluid contained in venturi reservoir 282 from easily passing through filter drain hole 288 in the direction of filter 287. In certain embodiments, the cross-sectional area of filter drain hole 288 is about 10% or less of the cross-sectional area of vacuum port 283.

Venturi reservoir 282 includes a level sensor area 289 defined in base 206. Vacuum port 283 is disposed above level sensor area 289. Level sensor area 289 is disposed along an optical path of an infrared light sensor in console 100 that is used to determine a fluid level in venturi reservoir 282. In certain embodiments, the infrared light sensor is a single camera sensor or a complementary metal oxide semiconductor (CMOS) sensor. During normal operation, a nominal fluid level in venturi reservoir 282 is between lower and upper limits of level sensor area 289. The opening corresponds to level sensor area 289. The relatively small size or footprint of level sensor area 289 enables the use of a more compact housing 205 compared to other designs.

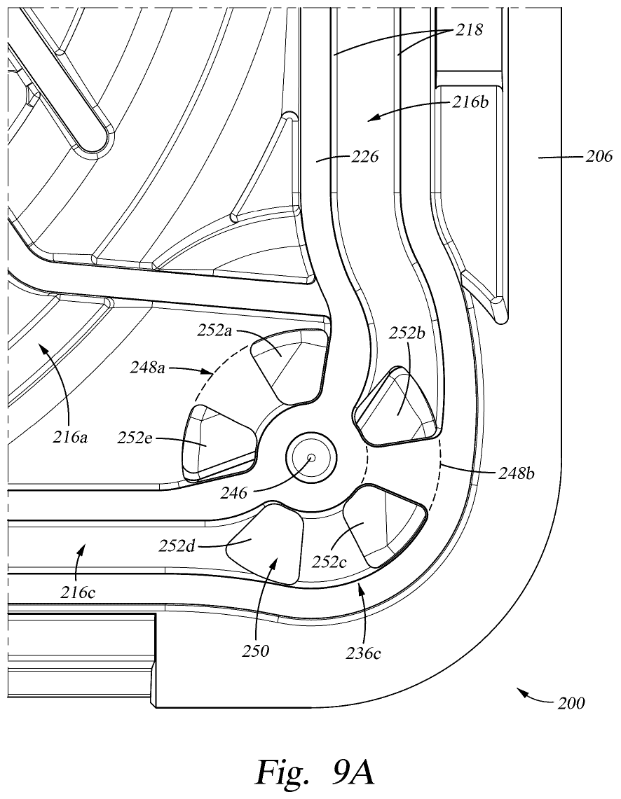

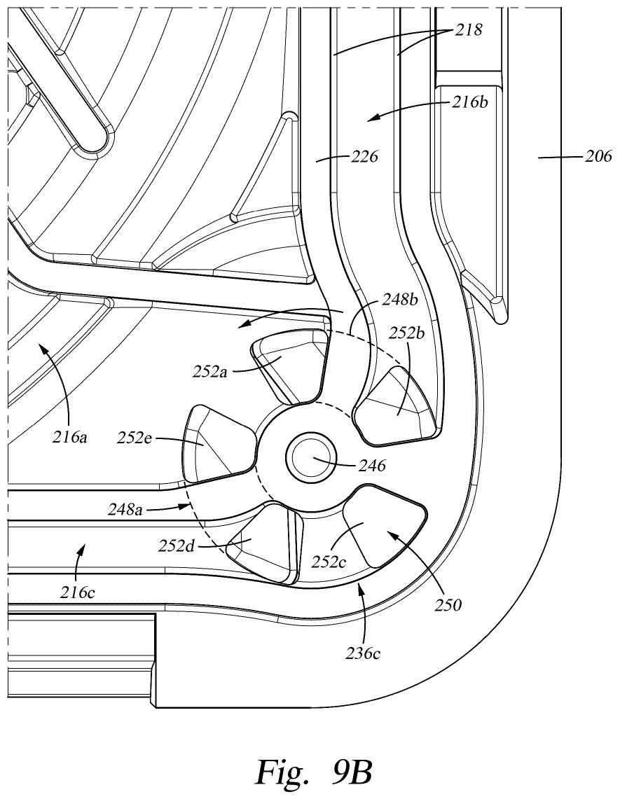

In certain embodiments, venturi reservoir 282 receives fluids that are aspirated through port 210a of base 206. For example, aspirated fluids may enter port 210a, flow through channel 216c to port 252d, and then enter into venturi reservoir 282 through port 252e. Thus, port 252e is in fluid communication with an aspiration line of surgical handpiece 112 (shown in FIG. 1A) through port 210a of base 206. Port 252e is disposed through base 206 below level sensor area 289. Note that ports 252d-eare also referred to as “fourth port” and “fifth port,” respectively, with regard to the description of the valve in conjunction with FIGS. 9A-9B.

In certain embodiments, venturi reservoir 282 receives fluids that are suctioned through port 292 on the front side of handle 208b. For example, fluids may enter port 292, flow through channel 293 in handle 208b to port 294a in base 206, and then enter into venturi reservoir 282 through port 294b. Port 294b is disposed through base 206 above level sensor area 289. In certain embodiments, fluids entering venturi reservoir 282 through port 252e or port 294b include a mixture of liquid, such as BSS (balanced salt solution), and air. In some other embodiments, the fluid is only liquid or only air. In certain embodiments, a flow rate of the fluid is about 200 cc/min (cubic centimeters per minute) or less.

The presence of air bubbles in or near level sensor area 289 interferes with accurate detection of the fluid level in venturi reservoir 282 because the air bubbles obscure the air-liquid interface. Therefore, in certain embodiments, a plurality of air baffles 295 (295a-c) are disposed within venturi reservoir 282 in order to divert air bubbles away from level sensor area 289. The plurality of air baffles 295 are integral with base 206 and contact cover 208a when cover 208a is coupled to base 206. In some other embodiments, the plurality of air baffles 295 are integral with cover 208a instead of base 206.

A lower air baffle 295a is disposed above port 252e and extends up and to the left of the viewer in FIG. 2E. An upper air baffle 295b is disposed below port 294b and extends down and to the left of the viewer in FIG. 2E. Air baffles 295a-b form corresponding channel-like structures within venturi reservoir 282 that begin at corresponding ports 252e, 294b and end on the left side of venturi reservoir 282. For example, first channel 216a corresponds to a portion of venturi reservoir 282 below lower air baffle 295a. A central air baffle 295c is located to the right of the respective ends of air baffles 295a-b and extends from below to above level sensor area 289. The position of central air baffle 295c prevents air bubbles from crossing level sensor area 289 even after the air bubbles pass above lower air baffle 295a and below upper air baffle 295b, respectively.

An opening between lower air baffle 295a and central air baffle 295c provides a path for equalization of fluid on the left and right sides of central air baffle 295c, so that the fluid level in level sensor area 289 corresponds to the actual fluid level in venturi reservoir 282. An opening between upper air baffle 295b and central air baffle 295c provides a path for the flow of air bubbles from the left side of central air baffle 295c to vacuum port 283 without crossing level sensor area 289.

A liquid baffle 296 is disposed within venturi reservoir 282 above level sensor area 289. Liquid baffle 296 is configured to reduce or prevent liquid from entering vacuum port 283 in cover 208a, such as during bubbling, frothing, or overrun of the liquid contained in venturi reservoir 282. Like air baffles 295, liquid baffle 296 is integral with base 206 and contacts cover 208a when cover 208a is coupled to base 206. An upper portion of central air baffle 295c extends directly below liquid baffle 296 so that liquid falling down from liquid air baffle 296 is blocked from entering the right side of central air baffle 295c and subsequently prevented from crossing level sensor area 289.

FIGS. 3A-3B are enlarged exploded front side and back side isometric views, respectively, of a portion of the surgical cassette 200 of FIG. 2A illustrating example valve assembly 204c having an upper valve elastomer and a lower valve elastomer, according to certain embodiments. FIGS. 4A-4E illustrate various views of the valve assembly 204c having the upper valve elastomer and the lower valve elastomer. Accordingly, FIGS. 3A-3B and 4A-4E are described together herein for clarity. Although described and depicted as valve assembly 204c, it should be understood that the valve assembly in FIGS. 3A-3B and 4A-4E may also be exemplary of one or more of the valve assemblies 204a, 204b, and 204d depicted in FIGS. 2A-2D.

Valve body 236c of third valve assembly 204c has a first end 240, a second end 242, a substantially cylindrical outer surface 244 connecting first end 240 and second end 242, and a longitudinal axis 246 orthogonal to first end 240. Cylindrical outer surface 244 includes multiple stepped portions having different outer dimensions. In some embodiments, the cylindrical surface includes a base portion 245 separated from a drive interface 258 by a first shoulder 262 and separated from a collar portion 247 by a second shoulder 249, the collar portion 247 having a larger diameter relative to the base portion 245 as best seen in FIG. 4E. In one embodiment, the base portion 245 has a diameter of, for example, approximately 13 millimeters (mm) and a longitudinal length of, for example, 5.21 mm, while the collar portion has a diameter of, for example, approximately 16 mm and a longitudinal length of, for example, 3.38 mm. Transitioning from collar portion 247 to a lower valve elastomer 251 is a third shoulder 259.

Valve body 236c is rotatable about longitudinal axis 246. Two passages 248 (248a-b) are shown as being formed in valve body 236c at second end 242. However, more or less passages 248 may be formed through valve body 236c in certain embodiments. In FIG. 3A, first passage 248a and second passage 248b are about equal in length when measured in a circumferential direction about longitudinal axis 246 (e.g., extending about longitudinal axis 246 in a circumferential direction by about 140° (degrees)-150°). In the illustrated embodiments, each passage 248 is sized to simultaneously open fluid communication with two ports of base 206 as described in more detail below with respect to FIGS. 9A-9B. In some other embodiments, each passage 248 may be sized to simultaneously open fluid communication with any suitable number of ports (e.g., two, three, or four ports). In the illustrated embodiments, passages 248 include arc-shaped annular segments extending circumferentially about longitudinal axis 246. In certain embodiments, a cross-section of passages 248 may be circular, round, oval, polygonal, square, any other suitable shape, or combinations thereof.

Terminal ends of each passage 248 are defined through first end 240 of valve body 236c. In certain embodiments, a center axis of each passage 248 at the terminal ends is parallel to longitudinal axis 246. In certain embodiments, at least a portion of each passage 248, e.g., the portion between the terminal ends, is orthogonal to longitudinal axis 246. In certain embodiments, during fabrication, passages 248 are machined or molded in a direction parallel to longitudinal axis 246, e.g., starting from second end 242. In other words, an entire surface of each passage 248 is visible from second end 242 when viewed in a direction parallel to longitudinal axis 246. In the illustrated embodiments, passages 248 include an equal flow area. In some other embodiments, passages 248 may have different flow areas. In certain other embodiments, there may only be one passage, or more than two passages (e.g., 2-3 passages, 3-4 passages, 4-5 passages, or 5-6 passages) formed in the valve body.

Five ports 252 are formed through base 206 as shown in FIG. 3B. In operation, valve body 236c is rotatable relative to the first side 234c of base 206 to align each passage 248 with a corresponding port 252 (252a-c) of base 206 to open pressure and/or fluid communication between corresponding ones of the plurality of channels 216. A flow axis through each port 252 is parallel to longitudinal axis 246 of valve body 236c. A shape of each port 252 may correspond to a cross-section of each passage 248 of valve body 236c to help maintain laminar flow therethrough. In certain embodiments, a cross-sectional shape of each passage 248 may be formed by continuing a shape of the corresponding port 252 as a swept surface through valve body 236c.

In the illustrated embodiments, five ports 252 are formed through base 206 within each bore 230. However, there may be any suitable number of ports (e.g., two to seven ports) in each bore 230. In the illustrated embodiments, ports 252 include arc-shaped annular segments. In some other embodiments, ports 252 may be circular, round, oval, polygonal, square, any other suitable shape, or combinations thereof. In the illustrated embodiments, ports 252 have an equal flow area. In some other embodiments, ports 252 may have different flow areas. In the illustrated embodiments, ports 252 are uniformly spaced in the circumferential direction. In some other embodiments, ports 252 may have different spacing in the circumferential direction.

Retaining ring 238c has an annular body 254 with a center opening 256. Retaining ring 238c fits over and around valve body 236c such that a drive interface 258 (shown in FIG. 3B) of valve body 236c is received within center opening 256. In some embodiments, drive interface 258 engages a drive mechanism of console 100 for rotating valve body 236c about longitudinal axis 246. Annular body 254 includes multiple stepped portions having different outer dimensions. As an example, the annular body 254 has an outer diameter (e.g., outer shoulder 260a) of approximately 18 mm and an inner diameter (e.g., inner shoulder 260b) of about 14 mm. At least one portion of annular body 254 is disposed radially between cylindrical outer surface 244 of valve body 236c and cylindrical inner wall 232c of bore 230c. At least another portion of annular body 254 is disposed outside bore 230c. An outer shoulder 260a formed between the stepped portions of annular body 254 contacts back side 212 of base 206 when retaining ring 238c is fully seated in bore 230c.

In certain embodiments, valve body 236c includes an orientation-identifying feature, or a hard stop feature, which can be used to correlate a rotational state of valve body 236c with one of the base 206 or retaining ring 238 in order to ensure proper alignment between passages 248 and corresponding ports 252 during operation. In FIGS. 3A-3B, the hard stop feature includes a first profile 264 formed on cylindrical outer surface 244 of valve body 236c which is configured to contact a corresponding second profile 266 formed on annular body 254 of retaining ring 238c. Further rotation of valve body 236c is prevented by contact between first profile 264 and corresponding second profile 266. As shown in FIG. 3B, second profile 266 is disposed in a corresponding notch 268 formed on cylindrical inner wall 232c of bore 230c so that rotational alignment of retaining ring 238c is fixed relative to base 206 which is necessary for functioning of the hard stop feature.

To correlate a rotational state of valve body 236c with retaining ring 238c using the orientation identifying feature, valve body 236c may be rotated in a first direction (e.g., clockwise) about longitudinal axis 246 until first profile 264 contacts a first side of second profile 266 at which point a first rotational state is recorded. Then, valve body 236c may be rotated in a second opposite direction (e.g., counterclockwise) about longitudinal axis 246 until first profile 264 contacts a second opposite side of second profile 266 at which point a second rotational state is recorded. Because rotational alignment of retaining ring 238c is fixed relative to base 206 via the second profile being inserted into notch 268, alignment between passages 248 and corresponding ports 252 is precisely known at any rotational state between the first and second rotational states corresponding to the hard stop feature. In some embodiments, the retaining rings 238 disclosed and described above in relation to the valve bodies 236c may also be used in conjunction with a single-passage valve body.

In some embodiments, the valve assemblies 204a-204d may comprise a combination of both multiple-passage valve bodies 236c and single-passage valve bodies. For example, first valve assembly 204a and third valve assembly 204c as seen in FIG. 2B may each comprise a multiple-passage valve body 236c which are each in pressure and/or fluid communication with first pump assembly 202a and port 210a to provide aspiration (suction) through port 210a and ventilation during an operation respectively, while the second valve assembly 204b and fourth valve assembly 204d each comprise a single-passage valve body which are each in pressure and/or fluid communication with second pump assembly 202b and port 210c to provide irrigation (infusion) through port 210c and bypass during the same operation, respectively.

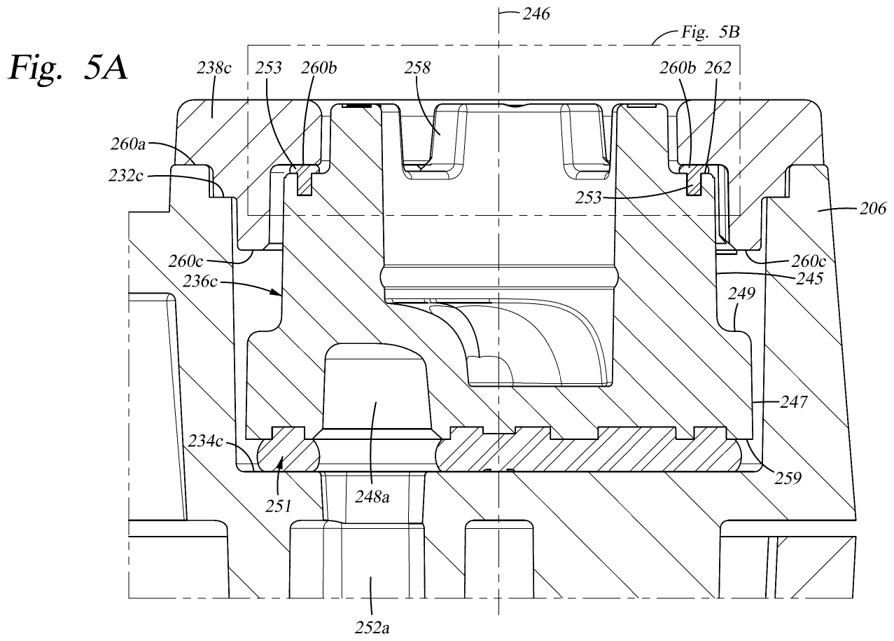

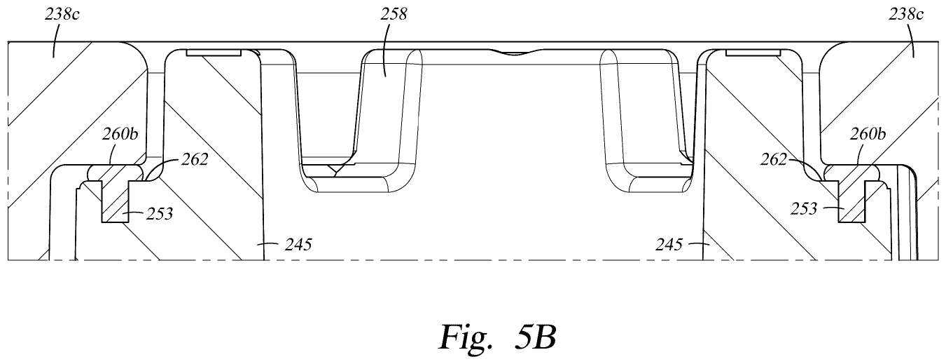

Valve assembly 204c includes a first, upper valve elastomer 253 (best seen in FIG. 4A) disposed between first end 240 of valve body 236c and retaining ring 238c (shown in FIGS. 3B and 5A-5B). Upper valve elastomer 253 rotatably contacts retaining ring 238c for facilitating a biasing force between the valve assembly 204c and the retaining ring 238c, which facilitates a fluidic seal provided by lower valve elastomer 251. Upper valve elastomer 253 has a first side 267 configured to engage with first shoulder 262 of valve body 236c to couple upper valve elastomer 253 to first end 240 of valve body 236c, and a second side 265 configured to movably engage with, or slide against, retaining ring 238c. As best shown in FIGS. 5A-5B, engagement or compression of upper valve elastomer 253 against retaining ring 238c and first shoulder 262 of valve body 236c creates an opposing biasing force in a direction towards first side 234c of base 206, such that the biasing force facilitates a fluidic seal at base 206 of housing 205.

In some embodiments, first side 267 is fixedly or movably coupled with first shoulder 262 of valve body 236c. In certain embodiments, upper valve elastomer 253 is formed from a rubber or other elastomeric material (e.g., silicone rubber) that is bonded (e.g., over-molded) onto valve body 236c at first end 240. In some other embodiments, valve body 236c and upper valve elastomer 253 may be integrally formed from the same material (e.g., a high density polyethylene or the like). In some embodiments, upper valve elastomer 253 is movably coupled to, or freely disposed against, first shoulder 262 of valve body 236c when valve assembly 204c is disposed in (e.g., assembled into) bore 230c of base 206 of surgical cassette 200.

Turning to FIG. 4E, in certain embodiments, first shoulder 262 of valve body 236c includes a recessed portion 280 configured to engage and/or align with a corresponding protruding portion 278 on first side 267 of upper valve elastomer 253 to couple upper valve elastomer 253 to valve body 236c. In certain embodiments, recessed portion 280 functions as an anchor that restrains protruding portion 278 when protruding portion 278 is formed during an over-molding process to fabricate upper valve elastomer 253 over valve body 236c, thereby bonding upper valve elastomer 253 and valve body 236c. In certain embodiments, recessed portion 280 may be continuously disposed or formed around first shoulder 262. In such an embodiment, a gap may exist between each edge (e.g., outer edge and inner edge) of upper valve elastomer 253 and first shoulder 262.

Other quantities and arrangements of recessed portions on first shoulder 262 and/or protruding portions on upper valve elastomer 253 are also contemplated for anchoring upper valve elastomer 253 to valve body 236c. For example, first shoulder 262 of valve body 236c may include one or more recessed portions (e.g., 3-4 recessed portions, 4-5 recessed portions, 6-7 recessed portions, or 7-8 recessed portions), such that upper valve elastomer 253 may have a corresponding number of protruding portions. Additionally, although protruding portion 278 of upper valve elastomer 253 and corresponding recessed portions 276 of valve body 236c are shown as being rectangular in FIG. 4E, the protruding portions and corresponding recessed portions may be circular, triangular, trapezoidal, any other suitable coupling shape, or combination thereof.

In some embodiments, some or all of recessed portion 280 may be discontinuous, such that one or more individual recessed portions may be placed at a location on first shoulder 262 (e.g., around perimeter of first shoulder 262). For example, first shoulder 262 of valve body 236c may include one recessed portion or more than two recessed portions (e.g., 2-3 recessed portions, 3-4 recessed portions, 4-5 recessed portions, or 5-6 recessed portions) spaced equally around the perimeter of first shoulder 262.

Second side 265 of upper valve elastomer 253 is configured to engage with retaining ring 238c to facilitate a fluidic seal at base 206 of housing 205. Second side 265 of upper valve elastomer 253, in certain embodiments, includes a rounded or semi-circular outer cross-sectional profile (shown in FIG. 4E) extending from an inner edge of upper valve elastomer 253 near drive interface 258 to an outer edge of upper valve elastomer 253. In some embodiments, second side 265 may have a rectangular, triangular, trapezoidal, or any other suitably shaped cross-sectional profile.

Upper valve elastomer 253 is generally continuous on first shoulder 262 and around drive interface 258 (shown in FIG. 4D). In certain embodiments, upper valve elastomer 253 has a continuous width around drive interface 258. In some embodiments, upper valve elastomer 253 may also be non-continuous around drive interface 258 and/or have a varying width around drive interface 258. For example, there may be more than one upper valve elastomer disposed around drive interface 258, such that a plurality of upper valve elastomers may be equidistantly disposed around drive interface 258 on first shoulder 262.

In some embodiments, upper valve elastomer 253 has a cross sectional width of, for example, 1.01 mm and an initial height of, for example, 0.50 mm which in certain embodiments may be compressible when a force is applied. In some embodiments, upper valve elastomer 253 has a cross sectional width equivalent to a cross sectional width of first shoulder 262.

In some embodiments, upper valve elastomer 253 may also be configured to engage with second shoulder 249 of valve body 236c. In such an embodiment, upper valve elastomer 253 has a cross sectional width of, for example, approximately 1.20 mm, which may be substantially equivalent to a cross sectional width of second shoulder 249. When upper valve elastomer 253 is configured to engage with second shoulder 249, protruding portion 278 may be received by a corresponding recessed portion of second shoulder 249.

Valve assembly 204c also includes a lower valve elastomer 251 (best seen in FIG. 4C) at second end 242 of valve body 236c which rotatably contacts first side 234c of base 206 (shown in FIGS. 3B and 5A) for scaling second end 242 with first side 234c. Lower valve elastomer 251 has a first side 270 coupled to second end 242 of valve body 236c, and a second side 272 configured to engage with first side 234c. Sealing between second end 242 and first side 234c of base 206 forms a sealing interface between the planar (e.g., non-cylindrical) surfaces of second end 242 and first side 234c. Because the sealing interface is on a longitudinal end (i.e., second end 242) of valve body 236c, this sealing arrangement may be referred to as end-scaling. In certain embodiments, lower valve elastomer 251 is formed from a rubber or elastomeric material (e.g., silicone rubber) which is bonded (e.g., over-molded) onto valve body 236c at second end 242. In some other embodiments, valve body 236c and lower valve elastomer 251 may be integrally formed from the same material (e.g., a high density polyethylene or the like).

Returning to FIG. 4E, in certain embodiments, second end 242 of valve body 236c includes recessed portions 276 (276a-b) configured to engage with corresponding protruding portions 274 (274a-b) on first side 270 of lower valve elastomer 251 to couple lower valve elastomer 251 to valve body 236c. In certain embodiments, recessed portions 276 function as anchors that restrain protruding portions 274 when protruding portions 274 are formed during an over-molding process to fabricate the lower valve elastomer 251 over valve body 236c, thereby bonding the lower valve elastomer 251 and valve body 236c.

In certain embodiments, the recessed portions 276 include a first, outer recessed portion 276a that may be continuously disposed or formed around a perimeter of second end 242. The recessed portions 276 may further include a second, inner recessed portion 276b that may be continuously disposed or formed around a lateral center point of second end 242 of valve body 236c. In some embodiments, first recessed portion 276a may be equidistant from second recessed portion 276b around an area between recessed portions 276a and 276b. In FIG. 4E, first recessed portion 276a is configured to engage with a first protruding portion 274a (outer portion) of lower valve elastomer 251, and second recessed portion 276b is configured to engage with a second protruding portion 274b (inner protruding portion) of lower valve elastomer 251.

Other quantities and arrangements of recessed portions on second end 242 and/or protruding portions on lower valve elastomer 251 are also contemplated for anchoring lower valve elastomer 251 to valve body 236c. For example, second end 242 of valve body 236c may include one or more recessed portions (e.g., 3-4 recessed portions, 4-5 recessed portions, 6-7 recessed portions, or 7-8 recessed portions), such that lower valve elastomer 251 may have a corresponding number of protruding portions. Additionally, although protruding portions 274 of lower valve elastomer 251 and corresponding recessed portions 276 of valve body 236c are shown as being rectangular in FIG. 4E, the protruding portions and corresponding recessed portions may be circular, triangular, trapezoidal, any other suitable coupling shape, or combination thereof. In some embodiments, recessed portions 276 can extend from the third shoulder 259 all the way to the second shoulder 249, such that recessed portions 276 may be through holes functioning as anchors which mechanically lock lower valve elastomer 251 to valve body 236 (i.e., opposed to a chemical bond).

In some embodiments, some or all of the recessed portions 276 may be discontinuous, such that one or more individual recessed portions may be placed at a location on second end 242 of valve body 236c (e.g., around perimeter of second end 242). For example, second end 242 of valve body 236c may include one recessed portion longitudinally opposite of interior surface 250 or two or more recessed portions spaced equally around the perimeter of second end 242. In some embodiments, the second end 242 of valve body 236c comprises one or more recessed portions laterally opposite of interior surface 250.

Second side 272 of lower valve elastomer 251 includes at least one interior surface 250. In some embodiments, interior surface 250 may be recessed from second side 272 of lower valve elastomer 251, such that the recessed surface is configured to prevent ballooning of lower valve elastomer 251 into ports 252 of base 206, which can cause wear and stress on lower valve elastomer 251, while also increasing torque. As such, a recessed interior surface reduces mechanical stress across the valve elastomer and other rigid components within valve assembly 204 throughout shelf life and during rotation of the valve assembly.

In certain embodiments, a lubricant (e.g. silicone oil) may also be used to facilitate relative rotation between interfacing surfaces of valve body 236c (e.g., first shoulder 262) and retaining ring 238c (e.g., inner shoulder 260b) as described in more detail with reference to FIG. 5B. In certain embodiments, lubricant may be dispensed onto one or more interfacing surfaces. As an example, lubricant is used to reduce torque and facilitate rotation of valve assembly 204c within bore 230 of housing 205. In such an example, the lubricant is also dispensed onto sealing interface between lower valve elastomer 251 of valve assembly 204c and the first side 234c of base 206.

In some embodiments, lower valve elastomer 251 can be impregnated with a lubricant (e.g., silicone oil) which eliminates the need of dispensing a lubricant on sealing surfaces. In other words, lower valve elastomer 251 is coated with a self-lubricating material (e.g., a slick silicone LSR (Liquid Silicone Rubber) coating) or is a self-lubricating silicone rubber. As such, lower valve elastomer 251 is configured to decrease a coefficient of friction.

Interior surface 250 is generally planar and orthogonal to longitudinal axis 246. Interior surface of lower valve elastomer 251 is adjacent to a passage (e.g., passage 248a or 248b) through lower valve elastomer 251 and valve body 236c. In certain embodiments, as shown in FIG. 4C, interior surface 250 of lower valve elastomer 251 is bounded by a first semi-circular inner edge 290a, two straight sides 290b from the inner edge 290a to an area of largest width 290c of interior surface 250, and a second semi-circular outer edge 290d. Interior surface 250 of lower valve elastomer 251 can extend about longitudinal axis 246 in either circumferential direction between about 20° and about 90° (e.g., between 35° and 75°). In some embodiments, interior surface 250 of lower valve elastomer 251 is surrounded by sealing portions 281 of second side 272 of lower valve elastomer 251 that extend past interior surface 250. In such an embodiment, interior surface 250 may not extend to a perimeter of lower valve elastomer 251.

When interior surface 250 is recessed from sealing portions 281, interior surface 250 may be defined as an area of an outer surface that is recessed from sealing portions 281 on second side 272 of lower valve elastomer 251. In some embodiments, interior surface 250 may be recessed from sealing portions 281 on second side 272 of lower valve elastomer 251 by a maximum height of, for example, 0.15 mm. As best seen in FIG. 5A, a center portion of lower valve elastomer 251 has a cross sectional width of, for example, 4.80 mm. In some embodiments, the passages 248 may be substantially identical and have cross sectional widths of, for example, 2.67 mm. In some embodiments, the lower valve elastomer 251 has an initial height of, for example, 1.52 mm which in certain embodiments may be compressible when a force is applied.

While interior surface 250 of lower valve elastomer 251 is shown as being a flat, linear surface in FIG. 4E, interior surface 250 may also be curved, angled relative to longitudinal axis 246, or any combination thereof. Additionally, there may be more than one interior surface (e.g., 2-3 interior surfaces, 4-5 interior surface, or 5-6 interior surfaces).

Greater detail of the interaction between upper valve elastomer 253 and retaining ring 238c is described with reference to FIGS. 5A-5B. When viewed in cross section, in an assembled state, upper valve elastomer 253 engages with and compresses between retaining ring 238c and first shoulder 262 of valve body 236c. This compression creates reactive forces in a direction toward first side 234c of base 206, which, in combination with opposing reactive forces provided by lower valve elastomer 251, facilitates a fluidic seal (e.g., fluidic sealing interface) at first side 234c of base 206 as detailed, for example, in FIG. 5A.

In certain embodiments, an inner shoulder 260b formed between the stepped portions of annular body 254 contacts upper valve elastomer 253 radially surrounding drive interface 258 to apply a biasing force on valve body 236c. In certain embodiments, a lubricant (e.g. silicone oil) may be used to facilitate relative rotation between interfacing surfaces of valve body 236c (e.g., first shoulder 262) and retaining ring 238c (e.g., inner shoulder 260b). In certain embodiments, lubricant may be dispensed onto one or more interfacing surfaces. In some embodiments, the valve elastomer 251 can be impregnated with a lubricant (e.g. silicone oil). In some embodiments, the lubricant may be any copolymer liquid comprising dimethylisiloxane and trifluoropropylmethylsiloxane that provides a lubricious coating.

The reactive forces created by upper valve elastomer 253 due to compression thereof against the retaining ring 238c are generally disposed in a direction parallel to longitudinal axis 246 (axially) of valve body 236c. Such reactive forces drive second end 242 of valve body 236c toward first side 234c of base 206, which compresses lower valve elastomer 251 against first side 234c of base 206, thereby forming a fluidic seal between passages 248 and corresponding ports 252.

In certain embodiments, lower valve elastomer 251 is compressed in the direction parallel to longitudinal axis 246 (axially) up to, for example, 34% of its total height, thereby compressing or reducing the overall height down to, for example, approximately 1 mm, when retaining ring 238c is fully seated in bore 230c. In certain embodiments, compression of lower valve elastomer 251 causes opposing reactive forces in the opposite direction, toward first end 240. In certain embodiments, retaining ring 238c is coupled to base 206 using a solid-state welding technique (e.g., ultrasonic welding) so that no gap or space is present between inner shoulder 260b of retaining ring 238c and upper valve elastomer 253. In some other embodiments, retaining ring 238c may be snap-fit, threaded, and/or adhered to base 206.

Implementing upper valve elastomer 253 in valve assembly 204c facilitates greater design and/or material selection flexibility as compared to other valve designs. For example, the downward reactive forces created by upper valve elastomer 253 enable the thickness of lower valve elastomer 251 to be reduced. Reducing the thickness of lower valve elastomer 251 may prevent lower valve elastomer 251 from ballooning into ports 252 during rotation of the valve assembly 204c, which reduces torque and abrasion to lower valve elastomer 251. Reducing the thickness of lower valve elastomer 251 may prevent lower valve elastomer 251 from ballooning outwards and contacting cylindrical inner wall 232, which reduces torque.

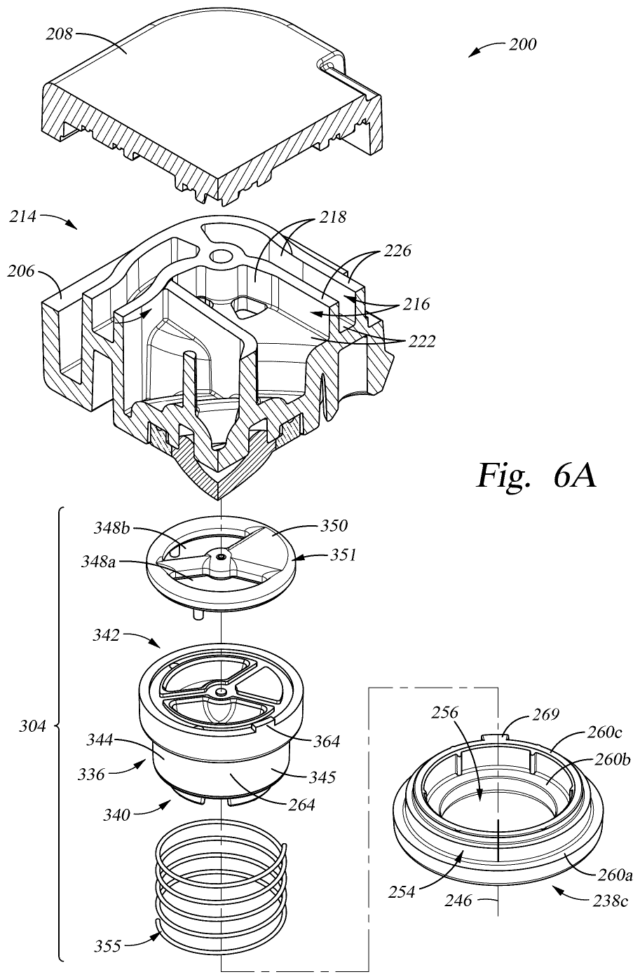

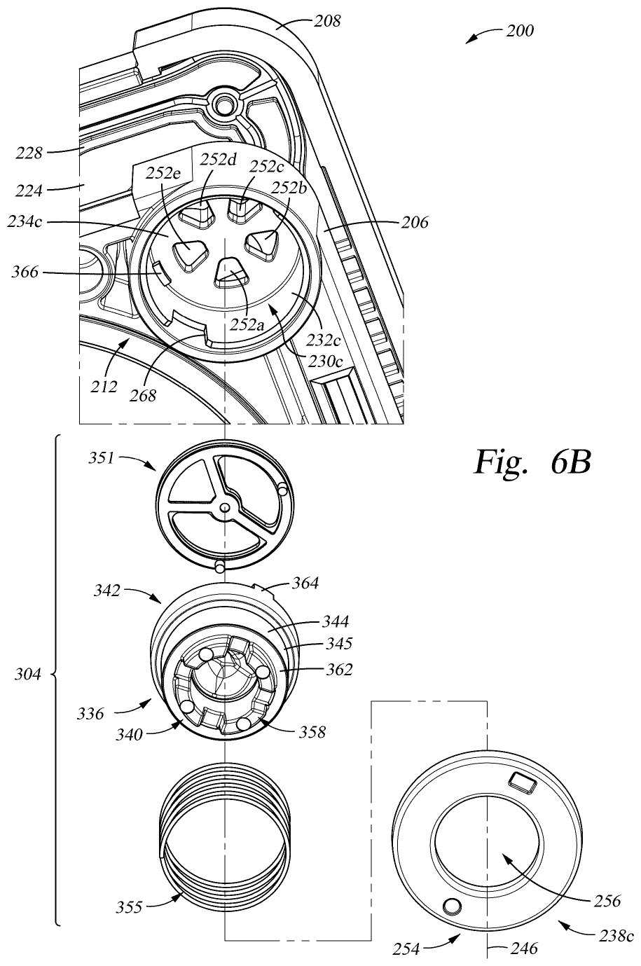

FIGS. 6A-6B are enlarged exploded front side and back side isometric views, respectively, of a portion of the surgical cassette 200 of FIG. 2A illustrating another example valve assembly 304 having a biasing member and a lower valve elastomer, according to certain embodiments. FIGS. 7A-7E illustrate various views of the valve assembly 304 having the biasing member and the lower valve elastomer. Accordingly, FIGS. 6A-6B and 7A-7E are described together herein for clarity. Note that the valve assembly 304 in FIGS. 6A-6B and 7A-7E may be exemplary of one or more of the valve assemblies 204a-204d depicted in FIGS. 2A-2D. Additionally, note that FIGS. 6A-6B, 7A-7E, and 8A-8B illustrate the portion of surgical cassette 200 and retaining ring 238c shown in FIGS. 3A-3B, 4A-4E, and 5A-5B. Thus, for purposes of clarity, details regarding surgical cassette 200 and retaining ring 238c are omitted below.

Valve assembly 304 may, in certain examples, be substantially similar to valve assembly 204c but for the inclusion of biasing member 355. For example, valve body 336 of third valve assembly 304 has a first end 340, a second end 342, a substantially cylindrical outer surface 344 connecting first end 340 and second end 342, and a longitudinal axis 246 disposed through the valve body 336 in an orientation orthogonal to first end 340. Cylindrical outer surface 344 includes multiple stepped portions having different outer dimensions. In some embodiments, as best seen in FIG. 7E, the cylindrical outer surface 344 includes a base portion 345 separated from a drive interface 358 by a first shoulder 362 and separated from a collar portion 347 by a second shoulder 349, the collar portion 347 having a larger diameter relative to the base portion 345. In one embodiment, the base portion 345 has a diameter of, for example, approximately 13 millimeters (mm) and a longitudinal length of, for example, 5.21 mm, while the collar portion has a diameter of, for example, approximately 16 mm and a longitudinal length of, for example, 3.38 mm. Transitioning from collar portion 347 to a lower valve elastomer 351 is a third shoulder 359.

Valve body 336 is rotatable about longitudinal axis 246. Two passages 348 (348a-b) are shown as being formed in valve body 336 at second end 342. However, more or less passages 348 may be formed through valve body 336 in certain embodiments. In FIG. 6A, first passage 348a and second passage 348b are about equal in length when measured in a circumferential direction about longitudinal axis 246 (e.g., extending about longitudinal axis 246 in a circumferential direction by about 140°- 150°). In the illustrated embodiments, each passage 348 is sized to simultaneously open fluid communication with two ports of base 206 as described in more detail below with respect to FIGS. 9A-9B. In some other embodiments, each passage 348 may be sized to simultaneously open fluid communication with any suitable number of ports (e.g., two, three, or four ports).

In the illustrated embodiments, passages 348 include arc-shaped annular segments extending circumferentially about longitudinal axis 246. In certain embodiments, a cross-section of passages 348 may be circular, round, oval, polygonal, square, any other suitable shape, or combinations thereof. Terminal ends of each passage 348 are defined through first end 340 of valve body 336. In certain embodiments, a center axis of each passage 348 at the terminal ends is parallel to longitudinal axis 246. In certain embodiments, at least a portion of each passage 348, e.g., the portion between the terminal ends, is orthogonal to longitudinal axis 246.

In certain embodiments, during fabrication, passages 348 are machined or molded in a direction parallel to longitudinal axis 246, e.g., starting from second end 342. In other words, an entire surface of each passage 348 is visible from second end 342 when viewed in a direction parallel to longitudinal axis 246. In the illustrated embodiments, passages 348 include an equal flow area. In some other embodiments, passages 348 may have different flow areas. In certain other embodiments, there may only be one passage, or more than two passages (e.g., 2-3 passages, 3-4 passages, 4-5 passages, or 5-6 passages) formed in the valve body.

Again, five ports 252 are formed through base 206 of surgical cassette 200 as shown in FIG. 6B. In operation, valve body 336 is rotatable relative to the first side 234c of base 206 to align each passage 348 with a corresponding port 252 (252a-e) of base 206 to open pressure and/or fluid communication between corresponding ones of the plurality of channels 216. A flow axis through each port 252 is parallel to longitudinal axis 246 of valve body 336. A shape of each port 252 may correspond to a cross-section of each passage 348 of valve body 336 to help maintain laminar flow therethrough. In certain embodiments, a cross-sectional shape of each passage 348 may be formed by continuing a shape of the corresponding port 252 as a swept surface through valve body 336.

In certain embodiments, valve body 336, like valve body 236c, includes an orientation-identifying feature, or a hard stop feature, which can be used to correlate a rotational state of valve body 336 with one of the base 206 or retaining ring 238c in order to ensure proper alignment between passages 348 and corresponding ports 252 during operation. In FIGS. 6A-6B, the hard stop feature includes a first profile 364 formed on valve body 336 which is configured to contact a corresponding bore profile 366 formed on first side 234c of base 206. As shown in FIG. 6B, a second profile 269 may be disposed in a corresponding notch 268 formed on cylindrical inner wall 232c of bore 230c so that rotational alignment of retaining ring 238c is fixed relative to base 206.

To correlate a rotational state of valve body 336 with retaining ring 238c using the hard stop feature 364, valve body 336 may be rotated in a first direction (e.g., clockwise) about longitudinal axis 246 until first profile 364 contacts a first side of bore profile 366 at which point a first rotational state is recorded. Then, valve body 336 may be rotated in a second opposite direction (e.g., counterclockwise) about longitudinal axis 246 until first profile 364 contacts a second opposite side of bore profile 366 at which point a second rotational state is recorded. Thus, alignment between passages 348 and corresponding ports 252 is precisely known at any rotational state between the first and second rotational states corresponding to the hard stop feature. In some embodiments, valve assembly 304 may not include a hard stop feature (e.g., may not include first profile 364). In some embodiments, the retaining rings 238 disclosed and described above in relation to the valve bodies 336 may also be used in conjunction with a single-passage valve body.

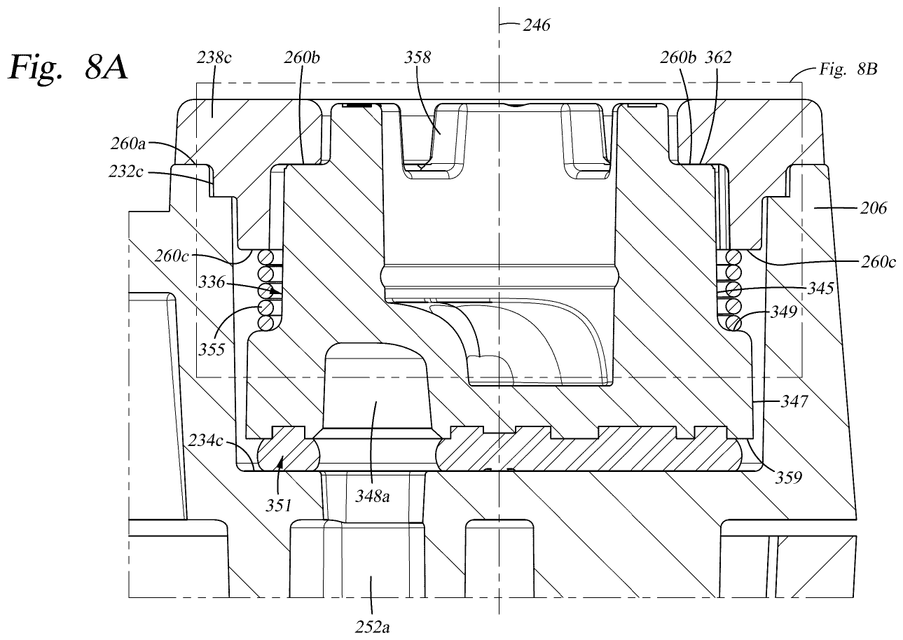

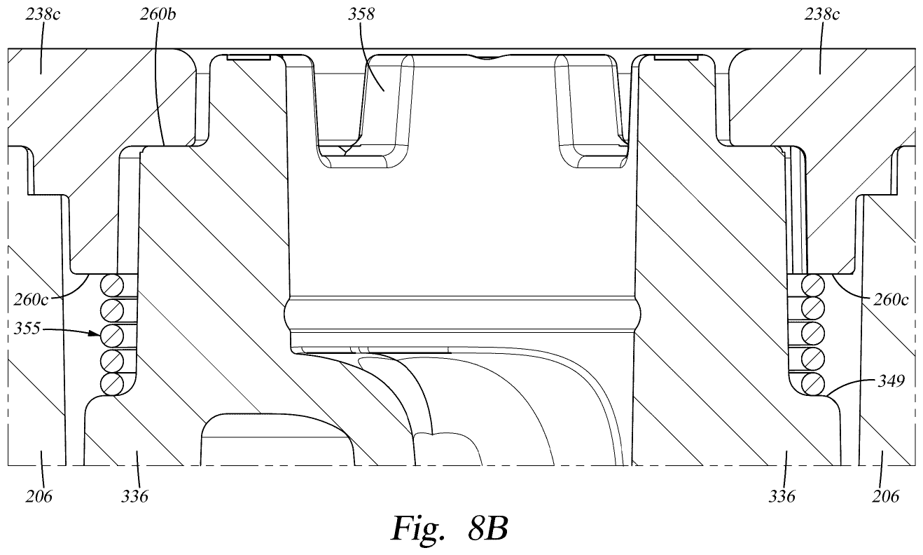

Valve assembly 304 includes a biasing member 355 (or a first sealing mechanism) (best seen in FIG. 7A) disposed around base portion 345 of valve body 336. Biasing member 355 may movably, and in some examples, rotatably, contact retaining ring 238c (shown in FIGS. 6B and 8A-8B) for facilitating a fluidic seal at base 206 of housing 205. In some examples, biasing member 355 may be fixedly attached to retaining ring 238c. Biasing member 355 has a first side 367 configured to engage with, or press and compress against, second shoulder 349 of valve body 336 to couple biasing member 355 to second end 342 of valve body 336, and a second side 365 configured to engage with retaining ring 238c. As best shown in FIGS. 8A-8B, the engagement or compression of biasing member 355 against retaining ring 238c and second shoulder 349 of valve body 336 creates a biasing force in a direction towards first side 234c of base 206, such that the biasing force facilitates a fluidic seal at base 206 of housing 205.

In some embodiments, biasing member 355 is a spring or other elastic device suitable for creating a biasing force and facilitating a fluidic seal at base 206. For example, in certain embodiments, biasing member 355 may include a compression spring, a helical spring, a disk spring, a leaf spring, a flat spring, or the like. In certain embodiments, biasing member 355 is formed from a metallic material, such as a metal alloy like stainless steel or the like. In certain embodiments, biasing member 355 is formed from a polymeric material, such as a thermoplastic polymer or other elastomeric polymer.

In some embodiments, first side 367 of biasing member 355 is fixedly coupled or movably coupled with second shoulder 349 of valve body 336. In certain embodiments, biasing member 355 is movably coupled to or freely disposed against second shoulder 349 of valve body 336 when valve assembly 304 is disposed in (e.g., assembled into) bore 230c of base 206 of surgical cassette 200.

In some embodiments, biasing member 355 may also be configured to engage with first shoulder 362 of valve body 336. When biasing member 355 is configured to engage with first shoulder 362, biasing member may be movably coupled or fixedly coupled to first shoulder 362.

Valve assembly 304 also includes a lower valve elastomer 351 (or second sealing mechanism) (best seen in FIG. 7C) at second end 342 of valve body 336 which rotatably contacts first side 234c of base 206 (shown in FIGS. 6B and 8A-8B) for scaling second end 342 with first side 234c. Lower valve elastomer 351 has a first side 370 coupled to second end 342 of valve body 336, and a second side 372 configured to engage with first side 234c. Sealing between second end 342 and first side 234c of base 206 forms a sealing interface between the planar (e.g., non-cylindrical) surfaces of second end 342 and first side 234c. Because the sealing interface is on a longitudinal end (i.e., second end 342) of valve body 336, this sealing arrangement may be referred to as end-scaling. In certain embodiments, lower valve elastomer 351 is formed from a rubber or elastomeric material (e.g., silicone rubber) which is bonded (e.g., over-molded) onto valve body 336 at second end 342. In some other embodiments, valve body 336 and lower valve elastomer 351 may be integrally formed from the same material (e.g., a high density polyethylene or the like).

Returning to FIG. 7E, in certain embodiments, second end 342 of valve body 336 includes recessed portions 376 (376a-b) configured to engage with corresponding protruding portions 374 (374a-b) on first side 370 of lower valve elastomer 351 to couple lower valve elastomer 351 to valve body 336. In certain embodiments, recessed portions 376 function as anchors that restrain protruding portions 374 when protruding portions 374 are formed during an over-molding process to fabricate the lower valve elastomer 351 over valve body 336, thereby bonding the lower valve elastomer 351 and valve body 336.

In certain embodiments, the recessed portions 376 include a first, outer recessed portion 376a that may be continuously disposed or formed around a perimeter of second end 342. The recessed portions 376 may further include a second, inner recessed portion 376b that may be continuously disposed or formed around a lateral center point of second end 342 of valve body 336. In some embodiments, first recessed portion 376a may be equidistant from second recessed portion 376b around an area between recessed portions 376a and 376b. In FIG. 7E, first recessed portion 376a is configured to engage with a first protruding portion 374a (outer portion) of lower valve elastomer 351, and second recessed portion 376b is configured to engage with a second protruding portion 374b (inner protruding portion) of lower valve elastomer 351.

Other quantities and arrangements of recessed portions on second end 342 and/or protruding portions on lower valve elastomer 351 are also contemplated for anchoring lower valve elastomer 351 to valve body 336. For example, second end 342 of valve body 336 may include one or more recessed portions (e.g., 3-4 recessed portions, 4-5 recessed portions, 6-7 recessed portions, or 7-8 recessed portions), such that lower valve elastomer 351 may have a corresponding number of protruding portions. Additionally, although protruding portions 374 of lower valve elastomer 351 and corresponding recessed portions 376 of valve body 336 are shown as being rectangular in FIG. 7E, the protruding portions and corresponding recessed portions may be circular, triangular, trapezoidal, any other suitable coupling shape, or combination thereof. In some embodiments, recessed portions 376 can extend from the third shoulder 359 all the way to the second shoulder 349, such that recessed portions 376 may be through holes functioning as anchors which mechanically lock lower valve elastomer 351 to valve body 336 (i.e., opposed to a chemical bond).

In some embodiments, some or all of the recessed portions 376 may be discontinuous, such that one or more individual recessed portions may be placed at a location on second end 342 of valve body 336 (e.g., around perimeter of second end 342). For example, second end 342 of valve body 336 may include one recessed portion longitudinally opposite of interior surface 350 or two or more recessed portions spaced equally around the perimeter of second end 342. In some embodiments, the second end 342 of valve body 336 comprises one or more recessed portions laterally opposite of interior surface 350.

Second side 372 of lower valve elastomer 351 includes at least one interior surface 350. In some embodiments, interior surface 350 may be recessed from second side 372 of lower valve elastomer 351, such that the recessed portion is configured to prevent ballooning of lower valve elastomer 351 into ports 252 of base 206, which can cause wear and physical stresses on lower valve elastomer 351 and other mating components, while also increase torque.

In certain embodiments, lubricant (e.g. silicon oil) may also be used to facilitate relative rotation between interfacing surfaces of valve body 336 (e.g., first shoulder 362) and retaining ring 238c (e.g., inner shoulder 260b). In certain embodiments, lubricant may be dispensed onto one or more interfacing surfaces. As an example, lubricant is used to reduce torque and facilitate rotation of valve assembly 304 within housing 205. In such an example, the lubricant is dispensed onto a sealing interface between lower valve elastomer 351 of valve assembly 304 and the first side 234(a-c) of base 206.

Interior surface 350 is generally planar and orthogonal to longitudinal axis 246. Interior surface 350 of lower valve elastomer 351 is adjacent to a passage (e.g., passage 348a or 348b) through lower valve elastomer 351 and valve body 336. In certain embodiments, as shown in FIG. 7D, interior surface 350 of lower valve elastomer 351 is bounded by a first semi-circular inner edge 390a, two straight sides 390b from the inner edge to an area of largest width 390c of interior surface 350, and a second semi-circular outer edge 390d. Interior surface 350 of lower valve elastomer 351 extends about longitudinal axis 246 in a circumferential direction by about 20° and about 90° (e.g., between 35° and 75°). In some embodiments, interior surface 350 of lower valve elastomer 351 is surrounded by sealing portions 381 of second side 372 of lower valve elastomer 351 that extend past interior surface 350. In such an embodiment, interior surface 350 may not extend to a perimeter of lower valve elastomer 351.

When interior surface 350 is recessed from sealing portions 381, interior surface 350 may be defined as an area of an outer surface that is recessed from sealing portions 381 on second side 372 of lower valve elastomer 351. In some embodiments, interior surface 350 may be recessed from sealing portions 381 on second side 372 of lower valve elastomer 351 by a maximum height of, for example, 0.152 mm and have a cross sectional width of, for example, 4.80 mm. In some embodiments, the passages 348 are substantially identical and have cross sectional widths of, for example, 2.67 mm. In some embodiments, the lower valve elastomer 351 has an initial height of, for example, 1.52 mm which in certain embodiments may be compressible when a force is applied.

While interior surface 350 of lower valve elastomer 351 is shown as being a flat, linear surface in FIG. 7E, interior surface 350 may also be curved, angled relative to longitudinal axis 246, or any combination thereof. Additionally, there may be more than one interior surface (e.g., 2-3 interior surfaces, 4-5 interior surfaces, or 5-6 interior surfaces). Greater detail of the interaction between biasing member 355 and retaining ring 238c is described with reference to FIGS. 8A-8B. When viewed in cross section, in an assembled state, biasing member 355 engages with and compresses against retaining ring 238c and second shoulder 349 of valve body 336. This engagement creates a biasing force in a direction toward first side 234c of base 206, which, in combination with forces provided by lower valve elastomer 351, facilitates a fluidic seal (e.g., fluidic sealing interface) at first side 234c of base 206 as detailed, for example, in FIG. 8B.

In certain embodiments, an interior shoulder 260c of retaining ring 238c, formed below inner shoulder 260b, contacts against biasing member 355 radially outward from base portion 345 of valve body 336 to facilitate the creation of a biasing force on valve body 336 by the biasing member 355. The biasing forces created by biasing member 355, which are generally reactive forces caused by the compression thereof against retaining ring 238c and first shoulder 262, are disposed in one or more directions parallel to longitudinal axis 246 (axially) of valve body 336. In certain embodiments, the compression of biasing member 355 causes reactive forces in opposing directions, toward first end 340 and second end 342. Such reactive forces drive second end 342 of valve body 336 toward first side 234c of base 206 which compresses lower valve elastomer 351 against first side 234c of base 206 thereby forming a seal between passages 348 and corresponding ports 252.

In certain embodiments, lower valve elastomer 351 is compressed in the direction parallel to longitudinal axis 246 (axially) up to, for example, 34% of its total height, thereby compressing or reducing the overall height down to, for example, approximately 1.02 mm, when retaining ring 238c is fully seated in bore 230c. In certain embodiments, compression of lower valve elastomer 351 causes opposing reactive forces in the opposite direction, toward first end 340. In certain embodiments, retaining ring 238c is coupled to base 206 using a solid-state welding technique (e.g., ultrasonic welding) so that no gap or space is present between inner shoulder 260b of retaining ring 238c and first shoulder 362 of valve body 336. In some other embodiments, retaining ring 238c may be snap-fit, threaded, and/or adhered to base 206.

Implementing biasing member 355 in valve assembly 304 facilitates greater design and/or material selection flexibility as compared to other valve designs. For example, the downward reactive forces created by biasing member 355 enable the thickness of lower valve elastomer 351 to be reduced. Reducing the thickness of lower valve elastomer 351 may prevent lower valve elastomer 351 from ballooning into ports 252 during rotation of valve assembly 304, which reduces torque and abrasion to lower valve elastomer 351. Further, using a thinner lower valve elastomer 351 may provide more material selection or design flexibility, allowing use of alternate materials that exhibit less compression set, or reaction force on rigid components.

In some embodiments, valve assembly 304 comprises biasing member 355, upper valve elastomer 253, and lower valve elastomer 351. In such an embodiment, biasing member 355 and upper valve elastomer 253 may collectively facilitate a fluidic seal at a base of the housing. For example, biasing member 355 and upper valve elastomer 253 facilitate the fluidic seal by engaging with interior shoulder 260c and inner shoulder 260b of retaining ring 238c, respectively. Other variations of such an embodiment may similarly be achieved.

FIGS. 9A-9B are front side elevation views of a portion of the surgical cassette of FIG. 2A illustrating two different valve positions, according to certain embodiments. In FIGS. 9A-9B, cover assembly 208 is omitted for clarity. Although FIGS. 9A-9B are described herein with reference to valve assembly 204c of FIGS. 3A-3B, 4A-4E, and 5A-5B, valve assembly 304 of FIGS. 6A-6B, 7A-7E, and 8A-8B may also employ similar features described herein.

A first valve rotational state of valve assembly 204c is illustrated in FIG. 9A. In the first valve rotational state, first passage 248a (indicated with dashed outline) of valve body 236c is aligned with first port 252a and fifth port 252e of venturi reservoir 282. When first passage 248a is aligned with both ports of venturi reservoir 282, fluid communication with venturi reservoir 282 is closed, and therefore, the first port 252a and fifth port 252e may be referred to as “closed” in the first valve rotational state. First port 252a is adjacent to fifth port 252e and is disposed through base 206 below level sensor area 289 like fifth port 252e as described above. In the first valve rotational state, second passage 248b (indicated with dashed outline) is aligned with a second port 252b corresponding to a second channel 216b and a third port 252c corresponding to a third channel 216c, thereby opening fluid communication between second channel 216b and third channel 216c. In the first valve rotational state, a flat segment of valve elastomer 251 in a clockwise direction relative to second passage 248b is aligned with a fourth port 252d, thereby closing fluid communication through fourth port 252d.

In the first valve rotational state, a hard prime may be performed on second channel 216b upstream of first pump assembly 202a (also referred to as an “aspiration path” of surgical cassette 200). In general, the hard prime involves building-up vacuum very high in the aspiration path and then suddenly opening the aspiration path to liquid from venturi reservoir 282. The sudden rush of liquid causes a high flow rate through the aspiration path and shears any trapped air bubbles from their positions. The trapped air bubbles are allowed to enter the main flow stream and are passed to a drain bag coupled to surgical cassette 200. In certain embodiments, the hard prime is an automated sequence performed by computer 103 of console 100. The hard prime may be performed as part of the surgical setup of surgical cassette 200. The hard prime provides lower and more repeatable total fluid compliance (improved vacuum responsiveness) of surgical cassette 200 and results in lower fluid volume surges during post-occlusion break surge events.

In certain embodiments, second channel 216b is in fluid communication with first pump assembly 202a (also referred to as an “aspiration pump”) and third channel 216c is in fluid communication with an aspiration line to a surgical site for aspirating fluid from the eye (e.g., via handpiece 112 shown in FIG. 1A). In such embodiments, the first rotational state corresponds to an aspiration state.

An example operation of valve assembly 204c is described below. In certain embodiments, occlusion or blockage of fluid flow through the handpiece can occur during aspiration due to build-up of surgical material (e.g., lens fragments) causing vacuum pressure to build-up between the handpiece and first pump assembly 202a. With sufficient vacuum pressure build-up, breakage of the occlusion often occurs leading to a rapid surge of fluid being aspirated from the eye and into valve assembly 204c through third channel 216c, and in certain embodiments, including second channel 216b and first pump assembly 202a. This process is commonly referred to as post-occlusion break surge.

The occurrence of post-occlusion break surge may be mitigated using certain embodiments disclosed herein. In certain embodiments, post-occlusion break surge is indicated by a rapid pressure change which may be detected using a pressure sensor in the handpiece or the surgical cassette. When post-occlusion break surge is detected, valve assembly 204c may be switched from the first valve rotational state of FIG. 9A to a second valve rotational state illustrated in FIG. 9B by rotating valve body 236c counterclockwise about longitudinal axis 246 by about 72 degrees (as indicated by the arrow).

In the second valve rotational state, first passage 248a (indicated with dashed outline) of valve body 236c is aligned with fourth port 252d and fifth port 252e, thereby opening fluid communication between venturi reservoir 282 and third channel 216c. In the second valve rotational state, second passage 248b (indicated with dashed outline) is aligned with first port 252a and second port 252b, thereby opening fluid communication between venturi reservoir 282 and second channel 216b. In the second valve rotational state, the flat segment of valve elastomer 251 in a clockwise direction relative to second passage 248b is aligned with third port 252c, thereby closing fluid communication through third port 252c.

In embodiments with second channel 216b in fluid communication with first pump assembly 202a and third channel 216c in fluid communication with the surgical site (as described above), the second rotational state corresponds to a dual path venting state. Thus, fluid communication is open between first channel 216a of venturi reservoir 282 and first pump assembly 202a through second channel 216b. In the dual path venting state, fluid communication is also open between first channel 216a of venturi reservoir 282 and the surgical site through third channel 216c.

Valve assembly 204c may be switched from the aspiration state shown in FIG. 9A to the dual path venting state shown in FIG. 9B by rotating valve body 236c counterclockwise about longitudinal axis 246 by about 72 degrees (as indicated by the arrow). Switching to the dual path venting state immediately when post-occlusion break surge is detected mitigates the resulting rapid aspiration of fluid from the eye by filling second channel 216b and third channel 216c with fluid before significant fluid flow from the eye occurs.

In the dual path venting state, first port 252a and fifth port 252e are opened simultaneously. Having first port 252a and fifth port 252e opened at the same time provides independent venting to venturi reservoir 282 of vacuum pressure built-up within corresponding channels 216b-c of base 206. For instance, vacuum pressure built-up in third channel 216c between fifth port 252e and the aspiration line is vented through fifth port 252e. Likewise, vacuum pressure built-up in second channel 216b between first port 252a and first pump assembly 202a is vented through first port 252a independently of the venting of the aspiration surgical site through the third channel 216c.

In certain embodiments, second channel 216b has a volume of about 4 cc (cubic centimeters), whereas the fluid line connection to the surgical site including third channel 216c has a volume of only about 0.5 cc. Thus, the relatively high compliance of second channel 216b contributes a majority of the vacuum pressure volume that leads to the post-occlusion break surge. Therefore, the independent venting that is provided by closing fluid communication between second channel 216b leading to the pump and third channel 216c leading to the eye greatly reduces the post-occlusion break surge volume.

In sum the valve designs described herein enable and/or facilitate greater design and/or material selection flexibility as compared to other valve designs, reduce compression set, stress relaxation, and physical stresses on valve assemblies, and/or help maintain sealing forces for longer periods of time. Additionally, torque and abrasion may be decreased.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A surgical cassette for use in ophthalmic surgical procedures, the surgical cassette comprising:
a housing;
a retaining ring coupled to the housing; and
a valve assembly coupled to the housing by the retaining ring and configured to control fluid communication of a channel within the housing, the valve assembly comprising:
a valve body having a first end with a first shoulder, a second end with a second shoulder, and a cylindrical surface connecting the first shoulder and the second shoulder; and
an upper valve elastomer comprising:
a first side of the upper valve elastomer configured to engage with the first shoulder of the valve body; and
a second side of the upper valve elastomer configured to engage with the retaining ring to facilitate a fluidic seal at a base of the housing.

2. The surgical cassette of claim 1, wherein engagement of the upper valve elastomer against retaining ring and the first shoulder of the valve body creates a biasing force in a direction toward the base of the housing, the biasing force facilitating the fluidic seal at the base of the housing.

3. The surgical cassette of claim 1, wherein:
the upper valve elastomer comprises a protruding portion on the first side of the upper valve elastomer; and
the valve body comprises a corresponding recessed portion on the first shoulder of the valve body, wherein the protruding portion is configured to engage with the corresponding recessed portion to couple the upper valve elastomer to the valve body.

4. The surgical cassette of claim 1, wherein the first side of the upper valve elastomer is fixedly coupled or movably coupled with the first shoulder of the valve body.

5. The surgical cassette of claim 1, wherein the valve assembly further comprises a lower valve elastomer comprising:
a first side of the lower valve elastomer configured to engage with the second end of the valve body; and
a second side of the lower valve elastomer configured to engage with the housing to provide the fluidic seal at the base of the housing.

6. A surgical cassette for use in ophthalmic surgical procedures, the surgical cassette comprising:
a housing;
a retaining ring coupled to the housing; and
a valve assembly coupled to the housing by the retaining ring and configured to control fluid communication of a channel within the housing, the valve assembly comprising:
a valve body having a first end with a first shoulder, a second end with a second shoulder, and a cylindrical surface connecting the first shoulder and the second shoulder; and
a biasing member disposed along the cylindrical surface of the valve body and configured to engage with the retaining ring to facilitate a fluidic seal at a base of the housing.

7. The surgical cassette of claim 6, wherein the biasing member creates a biasing force in a direction toward the base of the housing, the biasing force facilitating the fluidic seal at the base of the housing.

8. The surgical cassette of claim 6, wherein the biasing member is configured to engage with the second shoulder of the valve body.

9. The surgical cassette of claim 6, wherein the valve assembly further comprises a lower valve elastomer comprising:
a first side configured to engage with the second end of the valve body; and
a second side configured to engage with the housing to provide the fluidic seal at the base of the housing.

10. The surgical cassette of claim 9, wherein:
the lower valve elastomer comprises a protruding portion on the first side of the lower valve elastomer; and
the valve body comprises a corresponding recessed portion on the second end of the valve body, wherein the protruding portion is configured to engage with the corresponding recessed portion to couple the lower valve elastomer to the valve body.

11. A surgical cassette for use in ophthalmic surgical procedures, the surgical cassette comprising:
a housing;
a retaining ring coupled to the housing; and
a valve assembly coupled to the housing by the retaining ring and configured to control fluid communication of a channel within the housing, the valve assembly comprising:
a valve body having a first end with a first shoulder, a second end with a second shoulder, and a cylindrical surface connecting the first shoulder and the second shoulder;
a first sealing mechanism configured to engage with the retaining ring to facilitate a fluidic seal at a base of the housing; and
a second sealing mechanism configured to provide the fluidic seal at the base of the housing.

12. The surgical cassette of claim 11, wherein the first sealing mechanism comprises an upper valve elastomer or a biasing member.

13. The surgical cassette of claim 11, wherein the second sealing mechanism is a lower valve elastomer comprising:
a first side configured to engage with the second end of the valve body; and
a second side configured to engage with the housing to provide the fluidic seal at the base of the housing.

14. The surgical cassette of claim 13, wherein:
the lower valve elastomer comprises a protruding portion on the first side of the lower valve elastomer; and
the valve body comprises a corresponding recessed portion on the second end of the valve body, wherein the protruding portion is configured to engage with the corresponding recessed portion to couple the lower valve elastomer to the valve body.

15. The surgical cassette of claim 11, wherein the second sealing mechanism is configured to minimize ballooning into ports at the base of the housing.
